(19) United States
(12) Patent Application Publication
(10) Pub. No.: US 2005/0130917 A1
(43) Pub. Date: Jun. 16, 2005

(54) Gene expression during meningococcus adhesion

(75) Inventor: Guido Grandi, Milan (IT)

Correspondence Address: Chiron Corporation;Intellectual Property - R440, P.O. Box 8097, Emeryville, CA, 94662-8097, US

(73) Assignee: Chiron SRL, Siena (IT)

(21) Appl. No.: 10/481,456

(22) PCT Filed: Jun. 19, 2002
(86) PCT No.: PCT/IB02/03072

(51) Int. Cl. C12Q 1/68; G01N 33/554; G01N 33/569; C07H 21/04; A61K 48/00

(52) U.S. Cl. 514/44; 435/6; 435/7.32; 530/388.4; 536/23.7; 435/252.3

(57) ABSTRACT

The first step in human meningococcal infection involves adhesion to the epithelial cells of the nasopharynx tract. The invention provides various methods and compounds for preventing the attachment of Neisserial cells to epithelial cells and is based on the identification of 347 meningococcal genes which play a role in the adhesion process.

### CROSS REFERENCE TO RELATED APPLICATIONS

This application is a National Stage application of co-pending PCT application PCT/IB02/03072 filed Jun. 19, 2002, which was published in English under PCT Article 21(2) on Dec. 27, 2002, which claims the benefit of Great Britain application Serial No. GB0114940.0 filed Jun. 19, 2001. These applications are incorporated herein by reference in their entireties.

All documents cited herein are incorporated by reference in their entirety.

### TECHNICAL FIELD

This invention relates to gene expression in the bacterium Neisseria meningitidis, serogroup B (‘MenB’). In particular, it relates to the expression of genes when the bacterium binds to human epithelial cells.

### BACKGROUND ART

Neisseria meningitidis is a Gram-negative capsulated bacterium that colonises the epithelium of the human nasopharynx. Up to 30% of the human population asymptomatically carry the bacterium as well as other commensal Neisseria species such as N. lactamica. Through unkown mechanisms, N. meningitidis eventually spreads into the bloodstream and reaches the meninges, thus causing severe meningitis and sepsis in children [Merz & So (2000) Annu. Rev. Cell. Dev. Biol. 16, 423-457].

The current knowledge of the factors responsible for N. meningitidis pathogenesis derives from classical bacterial genetics and the application of a variety of in vitro and in vivo assays including the use of organ cultures and primary or immortalised cell lines. The advent of the genomics era has been used to investigate the host-pathogen interaction at molecular level. For example, Sun et al. [Nature Medicine (2000) 6:1269-73] used signature tagged mutagenesis to identify 73 genes whose inactivation confers an attenuated phenotype to N. meningitidis.

The first step in human MenB infection involves adhesion to the epithelial cells of the nasopharynx tract, and it is an object of the invention to facilitate the investigation and inhibition of this step.

### DISCLOSURE OF THE INVENTION

The invention provides methods for preventing the attachment of Neisserial cells to epithelial cells.

The invention is based on the identification of 347 MenB genes which play a role in the adhesion process. These genes are listed in Table I (up-regulated during adhesion) and Table II (down-regulated during adhesion). Furthermore, 180 of these genes (Table III) are absent in Neisseria lactamica, with the other 167 (Table IV) being found in both species.

Tables I to V refer to open reading frames using the “NMBnnnn” nomenclature of Tettelin et al. [Science (2000) 287:1809-1815]. These open reading frames are derived from a complete MenB genome sequence (strain MC58) and can be found in GenBank. It will be appreciated that the invention is not limited to using the precise MenB gene and protein sequences of Tettelin et al. but can be implemented by using related genes. For example, the invention may use genes from different strains within serogroup B [e.g. WO99/24578 and WO99/36544 give sequences from strain 2996] or from other serogroups of N. meningitidis [e.g. serogroup A—see Parkhill et al. (2000) Nature 404:502-506] or even from other Neisserial species [e.g. WO99/24578 and WO99/36544 give sequences from N. gonorrhoeae]. In general, therefore references to a particular MenB sequence should be taken to include sequences having identity thereto. Depending on the particular sequence, the degree of identity is preferably greater than 50% (e.g. 60%, 70%, 80%, 90%, 95%, 99% or more). This includes homologs, orthologs, allelic variants and mutants. Typically, 50% identity or more between two proteins may be considered to be an indication of functional equivalence. Identity between proteins is preferably determined by the Smith-Waterman homology search algorithm as implemented in the MPSRCH program (Oxford Molecular), using an affine gap search with parameters gap open penalty=12 and gap extension penalty=1. Collectively, these sequences are referred to herein as “adhesion-specific genes/proteins” (Table I & II), with the terms “adhesion-specific up-regulated genes/proteins” (Table I), “adhesion-specific down-regulated genes/proteins” (Table II), and “MenB-specific adhesion-specific genes/proteins” (Table III) also being used where appropriate.

Preferred adhesion-specific genes/proteins are from one of the following categories: Amino acid biosynthesis, Biosynthesis of cofactors, prosthetic groups, carriers, Cell envelope, Cellular processes, Central intermediary metabolism, DNA metabolism, Energy metabolism, Other categories, Protein fate, Protein synthesis, Regulatory functions, Transcription, Transport and binding proteins, Unknown function, Conserved hypothetical and hypothetical proteins. Genes/proteins involved in sulfur metabolism are particularly preferred.

Of the “adhesion-specific genes/proteins”, those in Table III are particularly preferred. Of the “adhesion-specific up-regulated genes/proteins”, those in Table V are particularly preferred.

References to a “Neisserial cell” below include any species of the bacterial genus Neisseria, including N. gonorrhoeae and N. lactamica. Preferably, however, the species is N. meningitidis. The N. meningitidis may be from any serogroup, including serogroups A, C, W135 and Y. Most preferably, however, it is N. meningitidis serogroup B.

References to an “epithelial cell” below include any cell found in or derived from the epithelium of a mammal. The cell may be in vitro (e.g. in cell culture) or in vivo. Preferred epithelial cells are from the nasopharynx. The cells are most preferably human cells.

Blocking the Neisseria-epithelium Interaction

The invention provides a method for preventing the attachment of a Neisserial cell to an epithelial cell, wherein the ability of one or more adhesion-specific protein(s) to bind to the epithelial cell is blocked.

The ability to bind may be blocked in various ways but, most conveniently, an antibody specific for the adhesion-specific protein is used.

The invention also provides antibody which is specific for an adhesion-specific protein. This antibody preferably has an affinity for the adhesion-specific protein of at least 10⁻⁷ M e.g. 10⁻⁸ M, 10⁻⁹ M, 10⁻¹⁰ M or tighter.

Antibodies for use in accordance with the invention may be polyclonal, but are preferably monoclonal.

It will be appreciated that the term “antibody” includes whole antibodies (e.g. IgG, IgA etc), derivatives of whole antibodies which retain the antigen-binding sites (e.g. F_{ab}, F_{ab′}, F_{(ab′)2} etc.), single chain antibodies (e.g. sFv), chimeric antibodies, CDR-grafted antibodies, humanised antibodies, univalent antibodies, human monoclonal antibodies [e.g. Green (1999) J Immunol Methods 231:11-23; Kipriyanov & Little (1999) Mol Biotechnol 12:173-201 etc.] and the like. Humanised antibodies may be preferable to those which are fully human [e.g. Fletcher (2001) Nature Biotechnology 19:395-96].

As an alternative to using antibodies, antagonists of the interaction between the MenB adhesion-specific protein and its receptor on the epithelial cell may be used. As a further alternative, a soluble form of the epithelial cell receptor may be used as a decoy. These can be produced by removing the receptor's transmembrane region and, optionally, cytoplasmic region [e.g. EP-B2-0139417, EP-A-0609580 etc.].

The antibodies, antagonists and soluble receptors of the invention may be used as medicaments to prevent the attachment of a Neisserial cell to an epithelial cell.

Inhibiting Expression of the Neisserial Gene

The invention provides a method for preventing the attachment of a Neisserial cell to an epithelial cell, wherein protein expression from one or more adhesion-specific gene(s) is inhibited. The inhibition may be at the level of transcription and/or translation.

A preferred technique for inhibiting expression of the gene is antisense [e.g. Piddock (1998) Curr Opin Microbiol 1:502-8; Nielsen (2001) Expert Opin Investig Drugs 10:331-41; Good & Nielsen (1998) Nature Biotechnol 16:355-358; Rahman et al. (1991) Antisense Res Dev 1:319-327; Methods in Enzmology volumes 313 & 314; Manual of Antisense Methodology (eds. Hartmann & Endres); Antisense Therapeutics (ed. Agrawal) etc.]. Antibacterial antisense techniques are disclosed in, for example, international patent applications WO99/02673 and WO99/13893.

The invention also provides nucleic acid comprising a fragment of x or more nucleotides from one or more of the adhesion-specific genes, wherein x is at least 8 (e.g. 8, 10, 12, 14, 16, 18, 20, 25, 30 or more). The nucleic acid will typically be single-stranded.

The nucleic acid is preferably of the formula 5′-(N)ₐ—(X)—(N)_{b}-3′, wherein 0≧a≧15, 0≧b≧15, N is any nucleotide, and X is a fragment of an adhesion-specific gene. X preferably comprises at least 8 nucleotides (e.g. 8, 10, 12, 14, 16, 18, 20, 25, 30 or more). The values of a and b may independently be 0, 1, 2, 3, 4, 5, 6, 7, 8, 9, 10, 11, 12, 13, 14 or 15. Each individual nucleotide N in the —(N)ₐ— and —(N)_{b}— portions of the nucleic acid may be the same or different. The length of the nucleic acid (i.e. a+b+length of X) is preferably less than 100 (e.g. less than 90, 80, 70, 60, 50, 40, 30 etc.).

It will be appreciated that the term “nucleic acid” includes DNA, RNA, DNA/RNA hybrids, DNA and RNA analogues such as those containing modified backbones (with modifications in the sugar and/or phosphates e.g. phosphorothioates, phosphoramidites etc.), and also peptide nucleic acids (PNA) and any other polymer comprising purine and pyrimidine bases or other natural, chemically or biochemically modified, non-natural, or derivatized nucleotide bases etc. Nucleic acid according to the invention can be prepared in many ways (e.g. by chemical synthesis, from genomic or cDNA libraries, from the organism itself etc.) and can take various forms (e.g. single stranded, double stranded, vectors, probes etc.).

The antisense nucleic acids of the invention may be used as medicaments to prevent the attachment of a Neisserial cell to an epithelial cell.

Knockout of the Neisserial Gene

The invention provides a method for preventing the attachment of a Neisserial cell to an epithelial cell, wherein one or more adhesion-specific gene(s) is knocked out.

The invention also provides a Neisseria bacterium in which one or more adhesion-specific gene(s) has been knocked out.

Techniques for producing knockout bacteria are well known, and knockout Neisseria have been reported [e.g. Moe et al. (2001) Infect. Immun. 69:3762-3771; Seifert (1997) Gene 188:215-220; Zhu et al. (2000) J. Bacteriol. 182:439-447 etc.].

The knockout mutation may be situated in the coding region of the gene or may lie within its transcriptional control regions (e.g. within its promoter).

The knockout mutation will reduce the level of mRNA encoding the corresponding adhesion-specific protein to <1% of that produced by the wild-type bacterium, preferably <0.5%, more preferably <0.1%, and most preferably to 0%.

The knockout mutants of the invention may be used as immunogenic compositions (e.g. as vaccines) to prevent Neisserial infection. Such a vaccine may include the mutant as a live attenuated bacterium.

Mutagenesis of the Neisserial Gene

The invention provides a method for preventing the attachment of a Neisserial cell to an epithelial cell, wherein one or more adhesion-specific gene(s) has a mutation which inhibits its activity.

The invention also provides a mutant protein, wherein the mutant protein comprises the amino acid sequence of an adhesion-specific protein, or a fragment thereof, but wherein one or more amino acids of said amino acid sequence is/are mutated.

The amino acids which is/are mutated preferably result in the reduction or removal of an activity of the adhesion-specific protein which is responsible directly or indirectly for adhesion to epithelial cells. For example, the mutation may inhibit an enzymatic activity or may remove a binding site in the protein.

The invention also provides nucleic acid encoding this mutant protein.

The invention also provides a method for producing this nucleic acid, comprising the steps of: (a) providing source nucleic acid encoding an adhesion-specific gene, and (b) performing mutagenesis (e.g. site-directed mutagenesis) on said source nucleic acid to provide nucleic acid encoding a mutant protein.

Mutation may involve deletion, substitution, and/or insertion, any of which may be involve one or more amino acids. As an alternative, the mutation may involve truncation.

Mutagenesis of virulence factors is a well-established science for many bacteria [e.g. toxin mutagenesis described in WO93/13202; Rappuoli & Pizza, Chapter 1 of Sourcebook of Bacterial Protein Toxins (ISBN 0-12-053078-3); Pizza et al. (2001) Vaccine 19:2534-41; Alape-Giron et al. (2000) Eur J Biochem 267:5191-5197; Kitten et al. (2000) Infect Immun 68:4441-4451; Gubba et al. (2000) Infect Immun 68:3716-3719; Boulnois et al. (1991) Mol Microbiol 5:2611-2616 etc.] including Neisseria [e.g. Power et al. (2000) Microbiology 146:967-979; Forest et al. (1999) Mol Microbiol 31:743-752; Cornelissen et al. (1998) Mol Microbiol 27:611-616; Lee et al. (1995) Infect Immun 63:2508-2515; Robertson et al. (1993) Mol Microbiol 8:891-901 etc.].

Mutagenesis may be specifically targeted to an adhesion-specific gene. Alternatively, mutagenesis may be global or random (e.g. by irradiation, chemical mutagenesis etc.), which will typically be followed by screening bacteria for those in which a mutation has been introduced into an adhesion-specific gene. Such screening may be by hybridisation assays (e.g. Southern or Northern blots etc.), primer-based amplification (e.g. PCR), sequencing, proteomics, aberrant SDS-PAGE gel migration etc.

The mutant proteins and nucleic acids of the invention may be used as immunogenic compositions (e.g. as vaccines) to prevent Neisserial infection.

Distinguishing Neisserial Species

The invention also provides methods for distinguishing Neisseria meningitidis from Neisseria lactamica based on the MenB-specific adhesion-specific genes and/or proteins of the invention.

Thus the invention provides a method for determining whether a Neisseria bacterium of interest is in the species meningitidis, comprising the step(s) of: (a) contacting the bacterium with a nucleic acid probe comprising the sequence of a MenB-specific adhesion-specific gene or a fragment thereof; and/or (b) contacting the bacterium with an antibody which binds to a MenB-specific adhesion-specific protein or an epitope thereof.

The method will typically include the further step of detecting the presence or absence of an interaction between the bacterium of interest and the MenB-specific nucleic acid or protein. The presence of an interaction indicates that the Neisseria of interest is of the species Neisseria meningitidis.

The bacterium of interest may be in a cell culture, for example, or may be within a biological sample believed or known to contain Neisseria. It may be intact or may be, for instance, lysed.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The method preferably confirms that the bacterium of interest is not Neisseria lactamica.

Investigating Neisseria

The invention also provides methods for determining where a Neisseria bacterium is within its infection cycle, comprising the step(s) of: (a) contacting the bacterium with a nucleic acid probe comprising the sequence of an adhesion-specific gene or a fragment thereof; and/or (b) contacting the bacterium with an antibody which binds to an adhesion-specific protein or an epitope thereof.

The method will typically include the further step of determining whether the probe or antibody has bound to the bacterium and to what extent. The method will generally also involve comparing the findings against a standard.

Preferably, the standard is a control value determined using a bacterium at a known stage in its infection cycle. It will be appreciated that the standard may have been determined before performing the method of the invention, or may be determined during or after the method has been performed. It may also be an absolute standard.

The invention also provides methods for assessing the likelihood that a Neisseria of interest is pathogenic, comprising the step(s) of: (a) contacting the bacterium with a nucleic acid probe comprising the sequence of an adhesion-specific gene or a fragment thereof; and/or (b) contacting the bacterium with an antibody which binds to an adhesion-specific protein or an epitope thereof. The method will typically include the further step of detecting the presence or absence of an interaction between the bacterium of interest and the adhesion-specific reagent. The presence of an interaction indicates that the Neisseria of interest is pathogenic.

The bacterium of interest may be in a cell culture, for example, or may be within a biological sample believed to containing Neisseria.

Screening Methods

The invention also provides methods for screening compounds to identify those (antagonists) which inhibit the binding of a Neisserial cell to an epithelial cell.

Potential antagonists for screening include small organic molecules, peptides, peptoids, polypeptides, lipids, metals, nucleotides, nucleosides, polyamines, antibodies, and derivatives thereof. Small organic molecules have a molecular weight between 50 and about 2,500 daltons, and most preferably in the range 200-800 daltons. Complex mixtures of substances, such as extracts containing natural products, compound libraries or the products of mixed combinatorial syntheses also contain potential antagonists.

Typically, an adhesion-specific protein of the invention is incubated with an epithelial cell and a test compound, and the mixture is then tested to see if the interaction between the protein and the epithelial cell has been inhibited.

Inhibition will, of course, be determined relative to a standard (e.g. the native protein/cell interaction). Preferably, the standard is a control value measured in the absence of the test compound. It will be appreciated that the standard may have been determined before performing the method, or may be determined during or after the method has been performed. It may also be an absolute standard.

The protein, cell and compound may be mixed in any order.

For preferred high-throughput screening methods, all the biochemical steps for this assay are performed in a single solution in, for instance, a test tube or microtitre plate, and the test compounds are analysed initially at a single compound concentration. For the purposes of high throughput screening, the experimental conditions are adjusted to achieve a proportion of test compounds identified as “positive” compounds from amongst the total compounds screened.

Other methods which may be used include, for example, reverse two hybrid screening [e.g. Vidal & Endoh (1999) TIBTECH 17:374-381] in which the inhibition of the Neisseria: receptor interaction is reported as a failure to activate transcription.

The method may also simply involve incubating one or more test compound(s) with an adhesion-specific protein of the invention and determining if they interact. Compounds that interact with the protein can then be tested for their ability to block an interaction between the protein and an epithelial cell.

The invention also provides a compound identified using these methods. These can be used to treat or prevent Neisserial infection. The compound preferably has an affinity for the adhesion-specific protein of at least 10⁻⁷ M e.g. 10⁻⁸ M, 10⁻⁹ M, 10⁻¹⁰ or tighter.

The Adhesion-specific Genes

The invention also provides adhesion-specific nucleic acid or protein of the invention for use as a medicament.

The invention also provides a nucleic acid array [e.g. Schena et al. (1998) TIBTECH 16:301-306; Ramsay (1998) Nature Biotech 16:40-44; Nature Genetics volume 21 (January 1999) supplement; Microarray Biochip Technology (ed. Schena) ISBN 1881299376; DNA Microarrays: A Practical Approach (ed. Schena) ISBN 0199637768], such as a DNA microarray, comprising at least 100 (e.g. 200, 300, or all 347) adhesion-specific nucleic acid sequences or fragments thereof. If fragments are used, these preferably comprise x or more nucleotides from the respective adhesion-specific gene, wherein x is at least 8 (e.g. 8, 10, 12, 14, 16, 18, 20, 25, 30, 35, 40 or more). The nucleic acid sequences on the array will typically be single-stranded.

Bacterial Vaccines

The invention provides GAPDH enzyme for use as a vaccine antigen for protecting or treating infection or disease caused by a Gram negative bacterium. The invention also provides the use of GAPDH enzyme in the manufacture of a vaccine for protecting or treating infection or disease caused by a Gram negative bacterium. The invention also provides a method for protecting or treating infection or disease caused by a Gram negative bacterium, comprising administering an immunogenic dose of GAPDH to a patient.

The invention provides N-acetylglutamate synthase enzyme for use as a vaccine antigen for protecting or treating infection or disease caused by a Gram negative or Gram positive bacterium. The invention also provides the use of N-acetylglutamate synthase enzyme in the manufacture of a vaccine for protecting or treating infection or disease caused by a bacterium. The invention also provides a method for protecting or treating infection or disease caused by a bacterium, comprising administering an immunogenic dose of N-acetylglutamate synthase to a patient.

The invention also provides a method for identifying a protein in a bacterium for use as a vaccine antigen, comprising: (a) identifying genes which are transcriptionally up-regulated in the bacterium during adhesion to a cell from a host which is susceptible to infection by the bacterium; and (b) identifying the protein encoded by said genes. Step (a) is conveniently performed using arrays.

Techniques

A summary of standard techniques and procedures which may be employed in order to perform the invention (e.g. to utilise the disclosed sequences for vaccination or diagnostic purposes) follows. This summary is not a limitation on the invention, but gives examples that may be used, but are not required.

General

The practice of the present invention will employ, unless otherwise indicated, conventional techniques of molecular biology, microbiology, recombinant DNA, and immunology, which are within the skill of the art. Such techniques are explained fully in the literature eg. Sambrook Molecular Cloning; A Laboratory Manual, Second Edition (1989) or Third Edition (2000); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

Definitions

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “comprising” means “including” as well as “consisting” e.g. a composition “comprising” X may consist exclusively of X or may include something additional e.g. X+Y.

The singular forms “a”, “and”, and “the” include plural referents unless the context clearly dictates otherwise. Thus, for example, reference to “a polynucleotide” includes a plurality of such polynucleotides and reference to “an epithelial cell” includes reference to one or more cells and equivalents thereof known to those skilled in the art, etc.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a Neisseria sequence is heterologous to a mouse host cell. A further examples would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (eg. 60%, 70%, 80%, 90%, 95%, 99% or more, calculated using the Smith-Waterman algorithm as described above). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic, variant typically encodes a protein having similar activity to that of the protein encoded by the gene to which it is being compared. An allelic variant can also comprise an alteration in the 5′ or 3′ untranslated regions of the gene, such as in regulatory control regions (eg. see U.S. Pat. No. 5,753,235).

Expression Systems

The Neisseria nucleotide sequences can be expressed in a variety of different expression systems; for example those used with mammalian cells, baculoviruses, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (eg. structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation [Sambrook et al. (1989) “Expression of Cloned Genes in Mammalian Cells.” In Molecular Cloning: A Laboratory Manual, 2nd ed.].

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter [Maniatis et al. (1987) Science 236:1237; Alberts et al. (1989) Molecular Biology of the Cell, 2nd ed.]. Enhancer elements derived from viruses may be particularly useful, because they usually have a broader host range. Examples include the SV40 early gene enhancer [Dijkema et al (1985) EMBO J. 4:761] and the enhancer/promoters derived from the long terminal repeat (LTR) of the Rous Sarcoma Virus [Gorman et al. (1982b) Proc. Natl. Acad. Sci. 79:6777] and from human cytomegalovirus [Boshart et al. (1985) Cell 41:521]. Additionally, some enhancers are regulatable and become active only in the presence of an inducer, such as a hormone or metal ion [Sassone-Corsi and Borelli (1986) Trends Genet. 2:215; Maniatis et al. (1987) Science 236:1237].

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation [Birnstiel et al. (1985) Cell 41:349; Proudfoot and Whitelaw (1988) “Termination and 3′ end processing of eukaryotic RNA. In Transcription and splicing (ed. B. D. Hames and D. M. Glover); Proudfoot (1989) Trends Biochem. Sci. 14:105]. These sequences direct the transcription of an mRNA which can be translated into the polypeptide encoded by the DNA. Examples of transcription terminater/polyadenylation signals include those derived from SV40 [Sambrook et al (1989) “Expression of cloned genes in cultured mammalian cells.” In Molecular Cloning: A Laboratory Manual].

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (eg. plasmids) capable of stable maintenance in a host, such as mammalian cells or bacteria. Mammalian replication systems include those derived from animal viruses, which require transacting factors to replicate. For example, plasmids containing the replication systems of papovaviruses, such as SV40 [Gluzman (1981) Cell 23:175] or polyomavirus, replicate to extremely high copy number in the presence of the appropriate viral T antigen. Additional examples of mammalian replicons include those derived from bovine papillomavirus and Epstein-Barr virus. Additionally, the replicon may have two replicaton systems, thus allowing it to be maintained, for example, in mammalian cells for expression and in a prokaryotic host for cloning and amplification. Examples of such mammalian-bacteria shuttle vectors include pMT2 [Kaufman et al. (1989) Mol. Cell. Biol. 9:946] and pHEBO [Shimizu et al. (1986) Mol. Cell. Biol. 6:1074].

The transformation procedure used depends upon the host to be transformed. Methods for introduction of heterologous polynucleotides into mammalian cells are known in the art and include dextran-mediated transfection, calcium phosphate precipitation, polybrene mediated transfection, protoplast fusion, electroporation, encapsulation of the polynulcleotide(s) in liposomes, and direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (eg. Hep G2), and a number of other cell lines.

ii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers & Smith, Texas Agricultural Experiment Station Bulletin No. 1555 (1987) (“Summers & Smith”).

Prior to inserting the DNA sequence encoding the protein into the baculovirus genome, the above described components, comprising a promoter, leader (if desired), coding sequence, and transcription termination sequence, are usually assembled into an intermediate transplacement construct (transfer vector). This may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extra-chromosomal element (e.g. plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (eg. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol. 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon, Maeda et al., (1985), Nature 315:592; human gastrin-releasing peptide, Lebacq-Verheyden et al., (1988), Molec. Cell. Biol. 8:3129; human IL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers & Smith supra: Ku et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skilled in the art. Namely, the plaques are screened under the light microscope for the presence (indicative of wild-type virus) or absence (indicative of recombinant virus) of occlusion bodies. “Current Protocols in Microbiology” Vol. 2 (Ausubel et al. eds) at 16.8 (Supp. 10, 1990); Summers & Smith, supra; Miller et al. (1989).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225). Cells and cell culture media are commercially available for both direct and fusion expression of heterologous polypeptides in a baculovirus/expression system; cell culture technology is generally known to those skilled in the art. See, eg. Summers & Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, eg. HPLC, affinity chromatography, ion exchange chromatography, etc.; electrophoresis; density gradient centrifugation; solvent extraction, etc. As appropriate, the product may be further purified, as required, so as to remove substantially any insect proteins which are also present in the medium, so as to provide a product which is at least substantially free of host debris, eg. proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iii. Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin; Gibberellins: in: Advanced Plant Physiology, Malcolm B. Wilkins, ed., 1984 Pitman Publishing Limited, London, pp. 21-52. References that describe other metabolically-regulated genes: Sheen, Plant Cell, 2:1027-1038 (1990); Maas et al., EMBO J. 9:3447-3452 (1990); Benkel and Hickey, Proc. Natl. Acad. Sci. 84:1337-1339 (1987).

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11 (2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, el al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacterial Systems

Bacterial expression techniques are known in the art. A bacterial promoter is any DNA sequence capable of binding bacterial RNA polymerase and initiating the downstream (3′) transcription of a coding sequence (eg. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al. (1984) Annu. Rev. Genet. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (trp) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The g-laotamase (bla) promoter system [Weissmann (1981) “The cloning of interferon and other mistakes.” In Interferon 3 (ed. I. Gresser)], bacteriophage lambda PL [Shimatake et al. (1981) Nature 292:128] and T5 [U.S. Pat. No. 4,689,406] promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid trp-lac promoter comprised of both trp promoter and lac operon sequences that is regulated by the lac repressor [A mann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EPO-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon [Shine et al. (1975) Nature 254:34]. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA.” In Biological Regulation and Development: Gene Expression (ed, R. F. Goldberger)]. To express eukaryotic genes and prokaryotic genes with weak ribosome-binding site [Sambrook et al. (1989) “Expression of cloned genes in Escherichia coli.” In Molecular Cloning: A Laboratory Manual].

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EPO-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene [Nagai et al. (1984) Nature 309:810]. Fusion proteins can also be made with sequences from the lacZ [Jia et al. (1987) Gene 60:197], trpE [Allen et al. (1987) J. Biotechnol. 5:93; Makoff et al. (1989) J. Gen. Microbiol. 135:11], and Chey [EP-A-0 324 647] genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (eg. ubiquitin specific processing-protease) to cleave the ubiquitin from the foreign protein. Through this method, native foreign protein can be isolated [Miller et al. (1989) Bio/Technology 7:698].

Alternatively, foreign proteins can also be secreted from the cell by creating chimeric DNA molecules that encode a fusion protein comprised of a signal peptide sequence fragment that provides for secretion of the foreign protein in bacteria [U.S. Pat. No. 4,336,336]. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb el al. (1984) EMBO J. 3:2437] and the E. coli alkaline phosphatase signal sequence (phoA) [Oka et al. (1985) Proc. Natl. Acad. Sci. 82:7212]. As an additional example, the signal sequence of the alpha-amylase gene from various Bacillus strains can be used to secrete heterologous proteins from B. subtilis [Palva et al. (1982) Proc. Natl. Acad. Sci. USA 79:5582; EP-A-0 244 042].

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (eg. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EP-A-0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Ann. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis [Palva et al. (1982) Proc. Natl. Acad. Sci. USA 79:5582; EP-A-0 036 259 and EP-A-063 953; WO 84/04541], Escherichia coli [Shimatake et al. (1981) Nature 292:128; Amann et al. (1985) Gene 40:123; Studier et al. (1986) J. Mol. Biol. 189:113; EP-A-0 036 776, EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. See eg. [Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus], [Miller et al. (1988) Proc. Natl. Acad. Sci. 85:856; Wang et al. (1990) J. Bacteriol. 172:949, Campylobacter], [Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids. In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus].

v. Yeast Expression

Yeast expression systems are also known to one of ordinary skill in the art. A yeast promoter is any DNA sequence capable of binding yeast RNA polymerase and initiating the downstream (3′) transcription of a coding sequence (eg. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PH05 gene, encoding acid phosphatase, also provides useful promoter sequences [Myanohara et al. (1983) Proc. Natl. Acad. Sci. USA 80:1].

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (EP-A-0 164 556). Furthermore, a yeast promoter can include naturally occurring promoters of non-yeast origin that have the ability to bind yeast RNA polymerase and initiate transcription. Examples of such promoters include, inter alia, [Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109;].

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See eg. EP-A-0 196 056. Another example is a ubiquitin fusion protein. Such a fusion protein is made with the ubiquitin region that preferably retains a site for a processing enzyme (eg. ubiquitin-specific processing protease) to cleave the ubiquitin from the foreign protein. Through this method, therefore, native foreign protein can be isolated (eg. WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the yeast invertase gene (EP-A-0 012 873; JPO. 62,096,086) and the A-factor gene (U.S. Pat. No. 4,588,684). Alternatively, leaders of non-yeast origin, such as an interferon leader, exist that also provide for secretion in yeast (EP-A-0 060 057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alphafactor. (eg. see WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (eg. plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 [Botstein et al. (1979) Gene 8:17-24], pC1/1 [Brake et al. (1984) Proc. Natl. Acad. Sci USA 81:4642-46461, and YRp17 [Stinchcomb et al. (1982) J. Mol. Biol. 158:157]. In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See eg. Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome [Orr-Weaver et al. (1983) Methods in Enzymol. 101:228-245]. An integrating vector may be directed to a specific locus in yeast by selecting the appropriate homologous sequence for inclusion in the vector. See Orr-Weaver et al,, supra. One or more expression construct may integrate, possibly affecting levels of recombinant protein produced [Rine et al. (1983) Proc. Natl. Acad. Sci. USA 80:6750], The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions [Butt et al. (1987) Microbiol, Rev. 51:351].

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts: Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis (Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol. 25:141], Pichia pastoris [Cregg, et al. (1985) Mol. Cell. Biol. 5:3376; U.S. Pat. Nos. 4,837,148 and 4,929,555], Saccharomyces cerevisiae [Hinnen et al. (1978) Proc. Natl. Acad. Sci. USA 75:1929; Ito et al. (1983) J. Bacteriol. 153:163], Schizosaccharomyces pombe [Beach and Nurse (1981) Nature 300:706], and Yarrowia lipolytica [Davidow, et al. (1985) Curr. Genet. 10:380471 Gaillardin, et al. (1985) Curr. Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed. See eg. [Kurtz et at. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al. (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 and 4,929,555; Pichia]; [Hinnen et al. (1978) Proc. Natl. Acad. Sci. USA 75;1929; Ito et al. (1983) J. Bacteriol. 153:163 Saccharomyces]; [Beach and Nurse (1981) Nature 300:706; Schizosaccharomyces]; [Davidow et al. (1985) Curr. Genet. 10:39; Gaillardin et al. (1985) Curr. Genet. 10:49; Yarrowia].

Antibodies

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

Antibodies against the proteins of the invention are useful for affinity chromatography, immunoassays, and distinguishing/identifying Neisseria proteins.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in viva immunization. Polyclonal antisera is obtained by bleeding the immunized animal into a glass or plastic container, incubating the blood at 25° C. for one hour, followed by incubating at 4° C. for 2-18 hours. The serum is recovered by centrifugation (eg. 1,000 g for 10 minutes). About 20-50 ml per bleed may be obtained from rabbits.

Monoclonal antibodies are prepared using the standard method of Kohler & Milstein [Nature (1975) 256:495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (eg. hypoxanthine, aminopterin, thymidine medium, “HAT”). The resulting hybridomas are plated by limiting dilution, and are assayed for production of antibodies which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the instant invention.

Pharmaceutical Compositions

Pharmaceutical compositions can comprise either polypeptides, antibodies, or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (eg. see WO98/20734), needles, and gene guns or hyposprays. Dosage treatment may be a single dose schedule or a multiple dose schedule.

See also Delivery Strategies for Antisense Oligonucleotide Therapeutics (ed. Akhtar) ISBN 0849347785.

Vaccines

Vaccines according to the invention may either be prophylactic (ie. to prevent infection) or therapeutic (ie. to treat disease after infection).

Such vaccines comprise immunising antigen(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59™ (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc; and (6) other substances that act as immunostimulating agents to enhance the effectiveness of the composition. Alum and MF59™ are preferred.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (MTP-PE), etc.

The immunogenic compositions (eg. the immunising antigen/immunogen/polypeptide/protein/nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, eg. by injection, either subcutaneously, intramuscularly, or transdermally/transcutaneously (eg. WO98/20734). Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

As an alternative to protein-based vaccines, DNA vaccination may be used [eg. Robinson & Torres (1997) Seminars in Immunol 9:271-283; Donnelly et al. (1997) Annu Rev Immunol 15:617-648; later herein].

Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in in vivo or ex viva modality. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153.

Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see O'Neill (1985) J. Virol. 53:160) polytropic retroviruses eg. MCF and MCF-MLV (see Kelly (1983) J. Virol. 45:291), spumaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (eg. HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J Virol 19:19-25), Abelson (ATCC No. VR-999), Friend (ATCC No. VR-245), Graffi, Gross (ATCC Nol VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936,WO94/03622,WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-967; Ram (1993) Cancer Res 53 (1993) 83-88; Takamiya (1992) J Neurosci Res 33:493-503; Baba (1993) J Neurosurg 79:729-735; Mann (1983) Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/11984, WO95/00655, WO95/27071, WO95/29993, WO95/34671, WO96/05320, WO94/08026, WO94/11506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curiel (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (ie. there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector. Construction of the Double-D ITR vector is disclosed in U.S. Pat. No. 5,478,745; Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar Institute), pHSVlac described in Geller (1988) Science 241:1667-1669 and in WO90/09441 and WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with the ATCC with accession numbers VR-977 and VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995, WO94/21792, WO92/10578, WO95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679640).

DNA vector systems such as eukaryotic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1110 and those described in Arnold (1990) J Cell Biochem L401; pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243; Kyzylagach virus, for example ATCC VR-927; Mayaro virus, for example ATCC VR-66; Mucambo virus, for example ATCC VR-580 and ATCC VR-1244; Ndumu virus, for example ATCC VR-371; Pixuna virus, for example ATCC VR-372 and ATCC VR-1245; Tonate virus, for example ATCC VR-925; Triniti virus, for example ATCC VR-469; Una virus, for example ATCC VR-374; Whataroa virus, for example ATCC VR-926; Y-62-33 virus, for example ATCC VR-375; O'Nyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curiel (1992) Hum Gene Ther 3:147-154 ligand linked DNA, for example see Wu (1989) J Biol Chem 264:16985-16987, eucaryotic cell delivery vehicles cells, for example see U.S. Ser. No. 08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/11033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamile, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin el al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033

Exemplary liposome and polycationic gene delivery vehicles are those described in U.S. Pat. Nos. 5,422,120 and 4,762,915; in WO 95/13796; WO94/23697; and WO91/14445; in EP-0524968; and in Stryer, Biochemistry, pages 236-240 (1975) W. H. Freeman, San Francisco; Szoka (1980) Biochem Biophys Acta 600:1; Bayer (1979) Biochem Biophys Acta 550:464; Rivnay (1987) Meth Enzymol 149:119; Wang (1987) Proc Natl Acad Sci 84:7851; Plant (1989) Anal Biochem 176:420.

A polynucleotide composition can comprises therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

Delivery Methods

Once formulated, the polynucleotide compositions of the invention can be administered (1) directly to the subject; (2) delivered ex vivo, to cells derived from the subject; or (3) in vitro for expression of recombinant proteins. The subjects to be treated can be mammals or birds. Also, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (eg. see WO98/20734), needles, and gene guns or hyposprays. Dosage treatment may be a single dose schedule or a multiple dose schedule.

Methods for the ex vivo delivery and reimplantation of transformed cells into a subject are known in the art and described in eg. WO93/14778. Examples of cells useful in ex vivo applications include, for example, stem cells, particularly hematopoetic, lymph cells, macrophages, dendritic cells, or tumor cells.

Generally, delivery of nucleic acids for both ex viva and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Polynucleotide and Polypeptide Pharmaceutical Compositions

The terms “polynucleotide” and “nucleic acid”, used interchangeably herein,

In addition to the pharmaceutically acceptable carriers and salts described above, the following additional agents can be used with polynucleotide and/or polypeptide compositions.

A. Polypeptides

One example are polypeptides which include, without limitation: asialoorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

B. Hormones, Vitamins, etc.

Other groups that can be included are, for example: hormones, steroids, androgens, estrogens, thyroid hormone, or vitamins, folic acid.

C. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

D. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) end neutral preparations. Cationic liposomes have been shown to mediate intracellular delivery of plasmid DNA (Felgner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner supra). Other commercially available liposomes include transfectace (DDAB/DOPE) and DOTAP/DOPE (Boerhinger). Other cationic liposomes can be prepared from readily available materials using techniques well known in the art. See, eg. Szoka (1978) Proc. Natl. Acad. Sci. USA 75:4194-4198; WO90/11092 for a description of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See eg. Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

E. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C & E, over time these lipoproteins lose A and acquire C & E. VLDL comprises A, B, C & E apoproteins, LDL comprises apoprotein B; and HDL comprises apoproteins A, C, & E.

The amino acid of these lipoproteins are known and are described in, for example, Breslow (1985) Annu Rev. Biochem 54:699; Law (1986) Adv. Exp Med. Biol. 151:162; Chen (1986) J Biol Chem 261:12918; Kane (1980) Proc Natl Acad Sci USA 77:2465; and Utermann (1984) Hum Genet 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mabey (1979) J Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443. Lipoproteins can also be purchased from commercial suppliers, such as Biomedical Techniologies, Inc., Stoughton, Mass., USA. Further description of lipoproteins can be found in Zuckermann et al. PCT/US97/14415.

F. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (X174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and purtrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene. Lipofectin™, and lipofectAMINE™ are monomers that form polycationic complexes when combined with polynucleotides/polypeptides.

Immunodiagnostic Assays

Neisseria antigens of the invention can be used in immunoassays to detect antibody levels (or, conversely, anti-Neisseria antibodies can be used to detect antigen levels). Immunoassays based on well defined, recombinant antigens can be developed to replace invasive diagnostics methods. Antibodies to Neisseria proteins within biological samples, including for example, blood or serum samples, can be detected. Design of the immunoassays is subject to a great deal of variation, and a variety of these are known in the art. Protocols for the immunoassay may be based, for example, upon competition, or direct reaction, or sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biolin and avidin, and enzyme-labeled and mediated immunoassays, such as ELISA assays.

Kits suitable for immunodiagnosis and containing the appropriate labeled reagents are constructed by packaging the appropriate materials, including the compositions of the invention, in suitable containers, along with the remaining reagents and materials (for example, suitable buffers, salt solutions, etc.) required for the conduct of the assay, as well as suitable set of assay instructions.

Use of Polypeptides to Screen for Peptide Analogs and Antagonists

Polypeptides encoded by the instant polynucleotides and corresponding full length genes can be used to screen peptide libraries to identify binding partners, such as receptors, from within the library. Peptide libraries can be synthesized according to methods known in the art (e.g. U.S. Pat. No. 5,010,175; WO91/17823). Agonists or antagonists of the polypeptides if the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a polypeptide binding partner, such as a receptor, encoded by a gene or a cDNA corresponding to a polynucleotide described herein, and at least one peptide agonist or antagonist of the binding partner. Such agonists and antagonists can be used to modulate, enhance, or inhibit receptor function in cells to which the receptor is native, or in cells that possess the receptor as a result of genetic engineering. Further, if the receptor shares biologically important characteristics with a known receptor, information about agonist/antagonist binding can facilitate development of improved agonists/antagonists of the known receptor.

Identification of Anti-bacterial Agents

Drug Screening Assays

Of particular interest in the present invention is the identification of agents that have activity in modulating expression of one or more of the adhesion-specific genes described herein, so as to inhibit infection and/or disease. Of particular interest are screening assays for agents that have a low toxicity for human cells.

The term “agent” as used herein describes any molecule with the capability of altering or mimicking the expression or physiological function of a gene product of a differentially expressed gene. Generally a plurality of assay mixtures are run in parallel with different agent concentrations to obtain a differential response to the various concentrations. Typically, one of these concentrations serves as a negative control i.e. at zero concentration or below the level of detection.

Candidate agents encompass numerous chemical classes, including, but not limited to, organic molecules (e.g. small organic compounds having a molecular weight of more than 50 and less than about 2,500 daltons), peptides, antisense polynucleotides, and ribozymes, and the like. Candidate agents can comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: polynucleolides, peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Screening of Candidate Agents In Vitro

A wide variety of in vitro assays may be used to screen candidate agents for the desired biological activity, including, but not limited to, labeled in vitro protein-protein binding assays, protein-DNA binding assays (e.g. to identify agents that affect expression), electrophoretic mobility shift assays, immunoassays for protein binding, and the like. For example, by providing for the production of large amounts of a differentially expressed polypeptide, one can identify ligands or substrates that bind to, modulate or mimic the action of the polypeptide. The purified polypeptide may also be used for determination of three-dimensional crystal structure, which can be used for modeling intermolecular interactions, transcriptional regulation, etc.

The screening assay can be a binding assay, wherein one or more of the molecules may be joined to a label, and the label directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assays described herein. Where the assay is a binding assay, these include reagents like salts, neutral proteins, e.g. albumin, detergents, etc. that are used to facilitate optimal protein-protein binding, protein-DNA binding, and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Many mammalian genes have homologs in yeast and lower animals. The study of such homologs' physiological role and interactions with other proteins in vivo or in vitro can facilitate understanding of biological function. In addition to model systems based on genetic complementation, yeast has been shown to be a powerful tool for studying protein-protein interactions through the two hybrid system.

Nucleic Acid Hybridisation

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. [supra] Volume 2, chapter 9, pages 9.47 to 9.57.

“Stringency” refers to conditions in a hybridization reaction that favor association of very similar sequences over sequences that differ. For example, the combination of temperature and salt concentration should be chosen that is approximately 120 to 200° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook el al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:
Tm=81+16.6(log₁₀Ci)+0.4[%(G+C)]−0.6(% formamide)−600/n−1.5 (% mismatch).
where Ci is the salt concentration (monovalent ions) and it is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (ie. stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double stranded complex, which is stable enough to be detected.

The nucleic acid probes will hybridize to the Neisseria nucleotide sequences of the invention (including both sense and antisense strands). Though many different nucleotide sequences will encode the amino acid sequence, the native Neisseria sequence is preferred because it is the actual sequence present in cells. mRNA represents a coding sequence and so a probe should be complementary to the coding sequence; single-stranded cDNA is complementary to mRNA, and so a cDNA probe should be complementary to the non-coding sequence.

The probe sequence need not be identical to the Neisseria sequence (or its complement)—some variation in the sequence and length can lead to increased assay sensitivity if the nucleic acid probe can form a duplex with target nucleotides, which can be detected. Also, the nucleic acid probe can include additional nucleotides to stabilize the formed duplex. Additional Neisseria sequence may also be helpful as a label to detect the formed duplex. For example, a non-complementary nucleotide sequence may be attached to the 5′ end of the probe, with the remainder of the probe sequence being complementary to a Neisseria sequence. Alternatively, non-complementary bases or longer sequences can be interspersed into the probe, provided that the probe sequence has sufficient complementarity with the a Neisseria sequence in order to hybridize therewith and thereby form a duplex which can be detected.

The exact length and sequence of the probe will depend on the hybridization conditions (e.g. temperature, salt condition etc.). For example, for diagnostic applications, depending on the complexity of the analyte sequence, the nucleic acid probe typically contains at least 10-20 nucleotides, preferably 15-25, and more preferably at least 30 nucleotides, although it may be shorter than this. Short primers generally require cooler temperatures to form sufficiently stable hybrid complexes with the template.

Probes may be produced by synthetic procedures, such as the triester method of Matteucci el at, [J. Am. Chem. Soc. (1981) 103:3185], or according to Urdea el al. [Proc. Natl. Acad. Sci. USA (1983) 80: 7461], or using commercially available automated oligonucleotide synthesizers.

The chemical nature of the probe can be selected according to preference. For certain applications, DNA or RNA are appropriate. For other applications, modifications may be incorporated eg. backbone modifications, such as phosphorothioates or methylphosphonates, can be used to increase in vivo half-life, alter RNA affinity, increase nuclease resistance etc. [eg. see Agrawal & Iyer (1995) Curr Opin Biotechnol 6:12-19; Agrawal (1996) TIBTECH 14:376-387]; analogues such as peptide nucleic acids may also be used [eg. see Corey (1997) TIBTECH 15:224-229; Buchardt et al. (1993) TIBTECH 11:384-386].

Alternatively, the polymerase chain reaction (PCR) is another well-known means for detecting small amounts of target nucleic acid. The assay is described in Mullis et al. [Meth. Enzymol. (1987) 155:335-350] & U.S. Pat. Nos. 4,683,195 & 4,683,202. Two “primer” nucleotides hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired Neisseria sequence.

A thermostable polymerase creates copies of target nucleic acids from the primers using the original target nucleic acids as a template. After a threshold amount of target nucleic acids are generated by the polymerase, they can be detected by more traditional methods, such as Southern blots. When using the Southern blot method, the labelled probe will hybridize to the Neisseria sequence (or its complement).

Also, mRNA or cDNA can be detected by traditional blotting techniques described in Sambrook et al [supra]. mRNA, or cDNA generated from mRNA using a polymerase enzyme, can be purified and separated using gel electrophotesis. The nucleic acids on the gel are then blotted onto a solid support, such as nitrocellulose. The solid support is exposed to a labelled probe and then washed to remove any unhybridized probe. Next, the duplexes containing the labeled probe are detected. Typically, the probe is labelled with a radioactive moiety.

### BRIEF DESCRIPTION OF DRAWINGS

FIG. 1 shows the adhesion kinetics of (1A) N. meningitidis and (1B) N. lactamica. The x-axis shows time in minutes and the y-axis shows bacterial colony forming units.

FIG. 2 is a representation of the whole microarray analysis of MenB and N. lactamica during interaction with 16HBE14 epithelial cells. FIGS. 2A & 2C show N. meningitidis data, and FIGS. 2B & 2D show N. lactamica data. In FIGS. 2A & 2B, the y-axis shows time in minutes and the x-axis is the number of regulated genes (285 for N. lactamica and 247 for N. meningitidis). In FIGS. 2C & 2D, the x-axis shows time in minutes and the y axis shows % of genes in particular categories (bottom=up-regulated; middle=no change; top=down-regulated).

FIG. 3 shows the pathways of sulfate and selenate up-take and metabolism in MenB. Genes involved in specific reactions and found up-regulated in adhering bacteria are boxed over the corresponding arrows.

FIG. 4 shows FACS analysis of four MenB proteins.

FIG. 5 shows FACS analysis of twelve MenB proteins. The maximal activation ratio (MAR) is boxed in each panel. The right-most line for the twelve proteins was obtained with adhering bacteria incubated with immune sera. The two left-most lines, which are often superimposed, were obtained with adhering and free bacteria incubated with pre-immune sera. The middle line was obtained with free bacteria incubated with immune sera.

FIG. 6 is a schematic representation of amino acid sequence variability within N. meningitidis of the five antigens reported in Table VII. The height of a line indicates the number of strains with an amino acid difference vs. MC58 at that particular amino acid residue. Strains used were: MC58, BZ83 and CU385 (cluster ET-5); 90/18311 and 93/4286 (cluster ET-37, serogroup C); 312294 (serogroup C) and 5/99 (cluster A4); M198172 (lineage 3), 2996, BZ232, 1000 (44, 14). As a control, MC58 PorA, a protein subject to gene variability, was compared for six strains (BZ83, 90/18311, 93/4286, 2996, BZ232, 1000).

### MODES FOR CARRYING OUT THE INVENTION

DNA microarrays carrying the entire gene repertoire of N. meningitidis serogroup B (strain MC58) have been used to analyse changes in gene expression induced in N. lactamica and MenB upon interaction with human 16HBE14 epithelial cells. Comparison of gene activation profiles in MenB and N. lactamica has identified genes regulated in both organisms and genes which are specific for MenB. This latter set of genes plays an important role in MenB virulence and pathogenicity.

Neisseria-epithelium Adhesion Kinetics

MenB MC58 and N. lactamica NL19 were grown on GCB agar (BD Biosciences, Franklin Lakes, N.J.) supplemented with 4 g/l glucose, 0.1 g/l glutamine, 2.2 mg/l cocarboxylase at 37° C. in 5% CO₂ for 16 hours. Adhesion assays were performed on 16HBE14, a polarized human bronchial epithelial cell line transformed with SV40 large T-antigen. Cells were cultured in D-MEM supplemented with 1.0% FCS, 1.5 mM glutamine and 100 μg/ml kanamycin sulfate.

Bacteria colonies from 16-hour old plates were suspended in D-MEM medium at a final OD₆₀₀ value of 1, and 0.4 ml of suspension (about 10⁹ bacteria) was added to epithelial cells (2×10⁶) and incubated at 37° C. in 5% CO₂ at different times. Cell-adhering bacteria were colony-counted after extensive washing (4 times) of epithelial cells with 5 ml HBSS-2% FCS (Life Technologies, Paisley, Scotland), followed by cell lysis with 1% saponin in HBSS for 10 minutes at 37° C. Non-specific binding of bacteria to plastic was estimated following the same procedure described above in the absence of epithelial cells.

Bacterial growth in D-MEM-10% FCS medium was determined by plating aliquots of the culture at different times ( ). To evaluate the growth rate of cell-adhering bacteria, both strains were incubated with HBE14 epithelial cells for 1 hour and non-adhering bacteria were removed by extensive washing. Fresh sterile medium was added and adhering bacteria were counted at different times after lysis of epithelial cells ( ). Finally, the kinetics of bacterial association was determined by adding bacteria to epithelial cells and cell-adhering bacteria were counted at different times after cell lysis ( ).

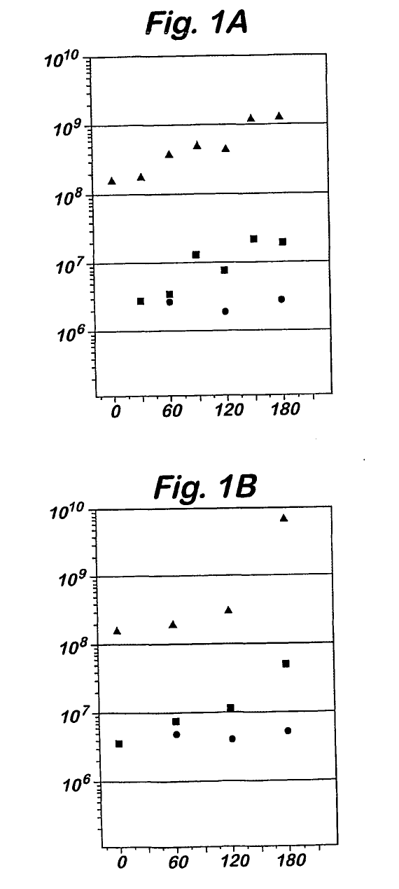

Cell samples were taken at time 0 and 30, 60, 120 and 180 minutes of co-cultivation. As shown in FIG. 1, adhesion kinetics were similar for the two bacteria. After 1 hour of co-cultivation, approximately 5-10 bacteria were found associated to each cell. This number increased with time, to reach 70-150 bacteria/cell after three hours, and paralleled the growth rate of MC58 in D-MEM culture medium. A large part of the time-dependent increase in cell-associated bacteria was due to new adhesion events taking place between cells and bacteria freely growing in the medium. When bacteria were incubated with the cells for 1 hour and the non-adhering bacteria were removed, the proliferation of cell-associated bacteria was negligible.

FACS Analysis

Adhering bacteria were collected after saponin treatment, washed with PBS-1% BSA and centrifuged. The bacterial capsule was permeabilized by dropwise addition of cold 70% EtOH directly on the pellet at −20° C. for 1 hour. Bacteria were washed, resuspended with PBS-1% BSA at the desired density and incubated either with sera of mice immunized with meningococcal recombinant proteins or with pre-immune sera [Pizza et al. (2000) Science, 287:1816-1820] for 2 hours on ice. After washing, bacteria were subsequently incubated with R-phycoerythrin-conjugated goat F(ab)₂ anti-mouse IgG (Jackson Immuno Research) for 1 hour on ice to detect antibody binding. Bacteria were washed and finally fixed with 0.25% para-formaldehyde and analyzed for cell-bound fluorescence using a FACScalibur flow cytometer (Becton Dikinson). Negative controls included non-infected human 16HBE14 epithelial cells subjected to the procedures described above.

Microarray Studies

DNA microarrays were prepared using DNA fragments of each annotated open reading frame (ORF) in the MenB MC58 genome [Tettelin et al.]. PCR primers were selected from a MULTIFASTA file of the genomic ORFs using either Primer 3 or Primer Premier (Premier Biosoft, Ca, USA) software, and the support of locally developed PERL scripts for handling multiple nucleotide sequence sets. The majority of PCR primer pairs were 17-25 nucleotides long and were selected within the ORFs sequences so as to have an average annealing temperature around 55° C. (range 50 to 60° C.) and produce amplified products of 250-1000 bp (when possible a length of 600-800 bp was selected). For ORFs shorter than 250 bp, primers annealing as close as possible to the start and stop codons were selected. In total, 2121 out of 2158 genes were amplified. The remaining 37 genes are duplicates, so 100% of the ORFs identified by Tettelin et al. were represented on the chips.

Amplification reactions were performed on MC58 genomic DNA with a Gene Amp PCR System 9700 (PE Applied Biosystems, Foster City, Calif.), using Taq polymerase (Roche Diagnostics, Mannheim, Germany) as recommended by the manufacturer. PCR products were purified using Qia-Quick spin columns (Qiagen, Chatsworth, Calif.) and quantified spectrophotometrically at OD₂₆₀.

Array printing was performed using a Gen III spotter (Amersham Pharmacia Biotech, Inc.) on type VII aluminum coated slides (Amersham Pharmacia Biotech, Inc.) according to the manufacturer's protocol. Thirty-seven different eukaryotic and prokaryotic genes were included in the chips as positive and negative controls. To establish the stringency of hybridization conditions, 6 sequences in the 73-100% homology range to a spiked control RNA were also included as controls. Hybridization conditions were set to detect hybridization signals of sequences having at least 73% homology.

Microarray analysis was carried out comparing the profile of total RNA extracted from bacteria growing in D-MEM-10% FCS culture medium (baseline control) and bacteria adhering to epithelial cells. Cell-adhering bacteria were prepared as described above. Total RNA was extracted from bacterial pellets using RNeasy spin columns (Qiagen, Chatsworth, Calif.). Bacterial RNA was quantified by one-step quantitative RT PCR of the 16S rRNA using LightCycler equipment (Roche Diagnostics). For RNA labeling, 1.5 μg were reverse transcribed using Superscript II™ reverse transcriptase (Life Technologies), random 9-mer primers and the fluorochromes Cy-3 dCTP and Cy-5 dCTP (Amersham Pharmacia Biotech, Inc.). Cy-3 and Cy-5 labelled cDNAs were co-purified on Qia-Quick spin columns (Qiagen). The hybridization probe was constituted by a mixture of the differently labeled cDNAs derived by cell-adhering bacteria and bacteria growing in liquid medium. Probe hybridization and washing were performed as recommended by the slide supplier (Amersham Pharmacia Biotech, Inc.). Slides were scanned with a GIII scanner (Amersham Pharmacia Biotech, Inc.) at 10 μm per pixel resolution. In each experiment the two RNA samples were labeled in the direct (Cy3-Cy5) and reverse (Cy5-Cy3) labeling reaction to correct for dye-dependent variation of labeling efficiency. The resulting 16-bit images were processed using the Autogene program (version 2.5, BioDiscovery, Inc., Los Angeles, Calif.). For each image, the signal value of each spot was determined by subtracting the mean pixel intensity of the background value, and normalizing to the median of all spot signals. The spots which gave a negative value after background subtraction were arbitrarily assigned the standard deviation value of negative controls. The data resulting from direct and reverse labeling were averaged for each spot. Expression ratios were obtained at each timepoint dividing hybridization signals from adhering bacteria RNA by non adhering bacteria RNA. The data of each timepoint represent the average of at least 4 independent experiments. Genes whose expression ratios changed by at least 2-fold (P-values<0,01) were considered up- or down-regulated. Expression pattern analysis and data visualization were done using GeneSpring software (version 3.1.0, Silicon Genetics, Redwood City, Calif.).

Panoramic View of Cell-Contact-Induced Changes in Gene Expression

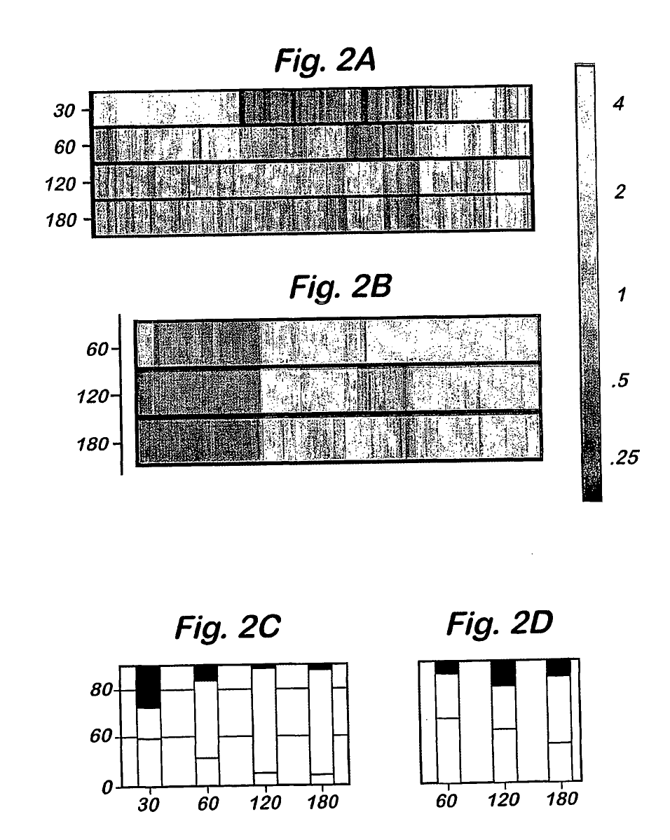

FIG. 2 is a color-code representation of the whole microarray analysis of MenB and N. lactamica during interaction with 16HBE14 epithelial cells. Panels a and b show clustered expression profiles of genes whose regulation differs from freely-growing bacteria by at least twofold at any timepoint. Panels c and d group the same regulated genes as in the panels a and b according to their activation state (up-regulated genes at the bottom of the columns, down-regulated genes at the top) to give a visual indication of the persistence of gene regulation.

Within 30 minutes of contact, 135 genes were up-regulated. For the majority of these genes, expression returned to baseline levels within 3 hours. Similarly, 118 genes were rapidly down-regulated, then slowly returned to pre-contact levels. A discrete number of genes, however, responded at later times suggesting secondary events. Only 8% of the regulated genes continued to maintain altered expression after 3 hours (FIG. 2C).

Overall, 347 genes altered their expression in MenB (and 285 in N. lactamica) by at least two-fold in at least one of the time-points analysed. Of these 347, 189 were up-regulated (Table I), 51 were down-regulated (Table II), and 7 were either up- or down-regulated depending on the analysis time point (included in Table I). MenB genes displaying expression differences higher than fourfold are reported in Table V.

Only 167 of the regulated genes (Table IV) were common to both bacteria, indicating that while a common set of genes responds to cell-contact, the different behavior of the two bacteria most likely resides in the 180 (Table III) and 118 genes specifically regulated in MenB and N. lactamica, respectively. When the chromosomal location of MenB-specific genes was analyzed, in a similar way to that reported for pathogenicity genes [Tettelin et al.], they were found evenly distributed throughout the genome with few striking exceptions.

Tettelin et al. had previously shown the existence of a cluster constituted by 37 perfectly duplicated genes. Seven out of these 37 are specifically activated in cell-adhering MenB: 6 genes belong to the sulfur acquisition and metabolism pathway (cysN-1 (NMB1153), cysH-1 (NMB1155), cysI-2 (NMB1189), cysJ-2 (NMB1190), cysD-2 (NMB1192), cysG-2 (NMB1194)) and the seventh, NMB1148, is classified in the ‘hypothetical gene’ family. Three additional duplicated genes also belonging to the ‘hypothetical gene’ family (NMB1128, NMB1167, NMB1187), were found activated in both Neisseria species. The concomitant duplication and activation of these genes is most likely indicative of their crucial role in the MenB infection process.

A relevant difference between MenB and N. lactamica is the time of persistence of RNA species in a cell-adhering population. A comparison of FIGS. 2a and 2b shows clearly that, while the number of regulated RNA species markedly decreased with time in MenB, 30% of the adhesion-specific N. lactamica RNAs remained regulated throughout the analysis and most of the regulated genes remained either in the activation or in the down-regulation state for a longer period of time.

The difference in mRNA levels between the two strains can be a consequence of different mechanisms of transcription regulation and/or RNA stability. Six transcription regulators were found regulated during adhesion in MenB as opposed to three (NMB1561, NMB1511 and crgA (NMB1856)) in N. lactamica. Furthermore, STM analysis by Sun et al. showed that inactivation of the RNAse genes NMB0686 and NMB0758 conferred an attenuated phenotype to MenB, suggesting the need of a rapid RNA turnover.

While the biological significance of the difference in RNA persistency between MenB and N. lactamica remains to be thoroughly investigated, the phenomenon may be linked to the different relationship the two bacteria have with the human host. N. lactamica has evolved to become a commensal and the nasopharyngeal epithelium represents its final destination. Therefore, once the bacterium comes into contact with epithelial cells, it would be expected that the program of RNA and protein synthesis remains essentially unaffected until substantial environmental variations occur. In contrast, MenB has the potential of moving from the epithelium to the endothelium and eventually of invading the blood stream and the meninges. This implicates a transient interaction with epithelial cells and a propensity to re-organize transcription and translation profiles to adapt itself rapidly to new environmental situations.

Cell Contact Induces Reduced Metabolism

The microarray analysis of the transcriptional events occurring after cell contact reveals that, in agreement with the growth reduction curve shown in FIG. 1, both N. meningitidis and N. lactamica reduce the activity of many growth-dependent genes. The list of down-regulated genes in MenB includes 34 genes involved in protein synthesis, 5 genes implicated in nucleotide synthesis and 7 genes of cell wall septation and synthesis. Reduction of transcription activity also involved the gene cluster encoding the ATP synthase F1 and F0 subunits (atpC (NMB1933), atpD (NMB1934), atpG (NMB1935), atpA (NMB1936), atpH (NMB1937), atpF (NMB1938), atpB (NMB1940)). This can be explained by an overall lower demand for ATP due to the reduced bacterial growth once associated to the cells or, alternatively, that bacteria are able, once cell-associated, to utilise part of the ATP synthesised by the host. Many of these metabolic genes (27 genes) were also down-regulated in N. lactamica, indicating that in both species the interaction with epithelial cells is at least partially mediated by similar events and a reduced metabolic demand.

Up-Regulation of Transporters

A second common event occurring in the two species appears to be the activation of some transport systems involved in transmembrane trafficking of different compounds. Commonly up-regulated transport machineries include the amino acid transporter gene NMB0177, the ABC transporters NMB0098 and NMB0041, the sulfate transporter gene cysT (NMB0881) and the ABC Fe³⁺ transporter gene NMB1990. Activation of genes involved in iron transport is intriguing, as the experimental conditions were not iron-limiting. Considering that, together with the ABC transporter gene, the transferrin binding protein gene (tbpI (NMB0461)) and the oxygen-independent coprophorphyrinogen III oxydase gene (NMB0665) were also activated in both species, the data suggest that, of the 3 possible iron acquisition pathways [Genco & Desai (1996) Trends in Microbiol. 4:179-184], adhering bacteria preferentially take up iron from transferrin.

Activation of transmembrane trafficking appears to be more pronounced in MenB. In fact, other transporter genes were specifically regulated in this organism and include the ABC cassette constituted by the 3 genes NMB0787, NMB0788, NMB0789, the amtB (NMB0615) transporter for ammonium, the ABC sulfate transporter (cysA (NMB0879), cysW (NMB0880), cysT (NMB0881), sbp (NMB1017)), the iron ABC transporter fbpA (NMB0634), the efflux pump gene NMB1719 and the chloride transporter gene NMB2006. NMB2006 is one of the 73 genes whose inactivation conferred an attenuated phenotype to MenB [Sun et al.]. Furthermore, activation of the sulfate transport system, which is strictly linked to sulfur-containing amino acid metabolism, is probably the most evident difference between cell-adhering MenB and N. lactamica.

Adhesion

In studying the biology of MenB invasion, a large number of experimental data have shown that after a first phase of localized adherence in which pili play an essential role, the genes of pili biosynthesis are down-regulated to allow intimate attachment and diffuse adherence [Pujol et al. (1997) Infect. Immun. 65:4836-42)]. The data described herein show that the pilE gene (NMB0018), whose product contributes to the interaction with epithelial cells and the induction of cortical plaques, was slightly up-regulated after 30 minutes. Furthermore, the pilC (NMB1847) transcript, encoding the major pilus adhesin involved in initial attachment to cells, was also marginally present in cell-associated bacteria after 30 minutes. However, at 30-minute incubation, crgA (NMB1856), the negative regulator of pilC1 expression [Deghmane et al. (2000) EMBO J. 19:1068-78], was already clearly up-regulated. In addition, pilT (NMB0052) RNA, whose product is responsible for pili retraction [Pujol et al. (1999) Proc. Nat. Acad. Sci. U.S.A. 96:4017-22], although not up-regulated, was one of the most abundant RNA species among total bacterial RNAs. As for the other pili genes, they appeared poorly transcribed and pilP (NMB1811) was down-regulated. Surface polysaccharide genes were transiently activated at initial contact, but then rapidly returned to baseline levels.

Intimate attachment requires the involvement of membrane-associated proteins interacting with specific cellular receptors. Several bacterial proteins have been proposed, the best candidates being the Opa/Opc proteins, porins and adhesins. The microarray data on MenB show that the opa/opc genes and the porin genes were not regulated during adhesion but were very actively transcribed throughout the three-hour incubation. Furthermore, MafA adhesins (mafA-1 (NMB0375), mafA-2 (NMB0652)) were up-regulated at the beginning of our kinetics analysis and the macrophage infectivity potentiator (MIP)-related protein (NMB0995) was constantly up-regulated. The expression of MIP genes is characteristic of intracellular pathogens and is known to increase their survival inside infected host cells [Susa et al. (1996) Infect. Immun. 64:1679-1684; Wintemeyer et al. (1995) Infect. Immun. 63:4576-4583; Horne et al. (1997) Infect. Immun. 65:806-810].

When expression of adhesion genes was analyzed in N. lactamica, a similar transcriptional pattern was observed, except that mafA-1 is MenB-specific. Therefore, apart from mafA-1 (and few additional pilin genes specific for N. lactamica), the overall expression profile would indicate that the two bacterial species utilise similar mechanisms of adhesion to epithelial cells.

Up-regulation of Amino Acid and Selenocysteine Biosynthesis

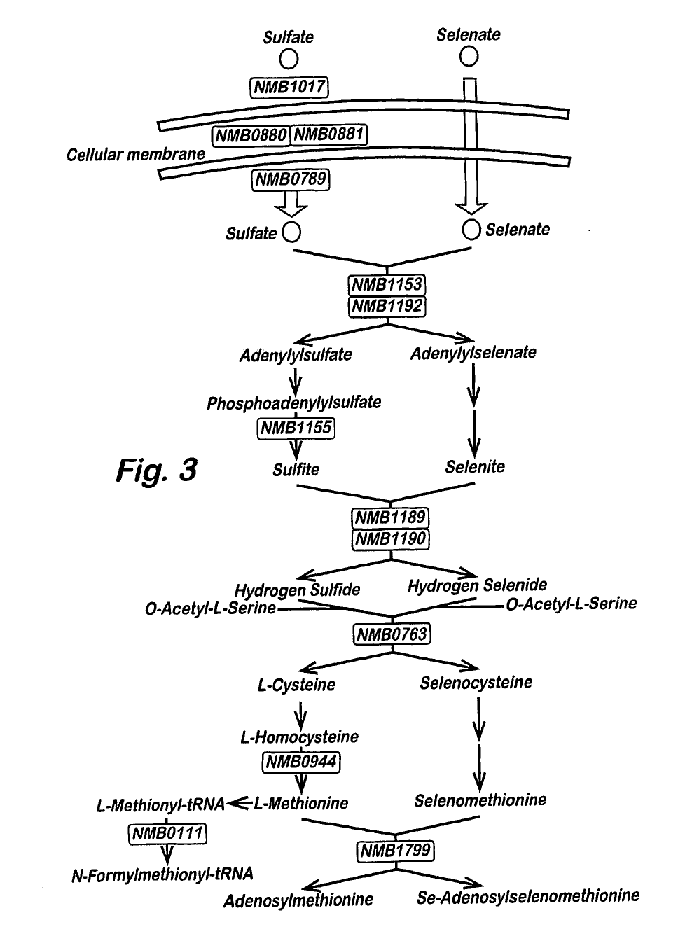

In vivo expression technology (IVET) [Mahan et al. (1993) Science 259:686-688] and signature tagged mutageneis (STM) [Hensel et al. (1995) Science 269:400-403] have shown that amino acid metabolism plays an important role in the infective process of many pathogens, including Staphylococcus aureus, Pseudomonas aeruginosa, Streptococcus pneumoniae, Salmonella typhimurium, and Brucella suis [Shea et al. (2000) Curr. Opin. Microbiol. 3:451-458]. In agreement with these observations, this microarray analysis indicates that 16HBE14-associated MenB and N. lactamica upregulated some of the genes involved in the synthesis of several amino acids. In MenB, a more pronounced activation involves histidine, methionine, cysteine and their seleno-derivatives. Overall, 17 genes (including sulfate uptake genes) are implicated in the synthesis of adenosylmethionine, methionine and N-formylmethionyl-tRNA (FIG. 3). Considering that 13 of these genes were up-regulated together with the siroheme synthase gene ((cysG-2) NMB1194, siroheme is the cofactor of sulfite reductase), the data unambiguously indicate that sulfur acquisition and metabolism play a key role in the adhesion process of MenB and represent one of the most striking metabolic differences between the two adhering bacteria.

Hypothetical Proteins

The most represented gene family responding to cell contact is the family of genes coding for ‘hypothetical proteins’ (107 genes in MenB, 54 of which also in N. lactamica). The 53 genes specifically induced in N. meninigitidis are likely to play a role in virulence.

Glyceraldehyde 3-phosphate Dehydrogenase

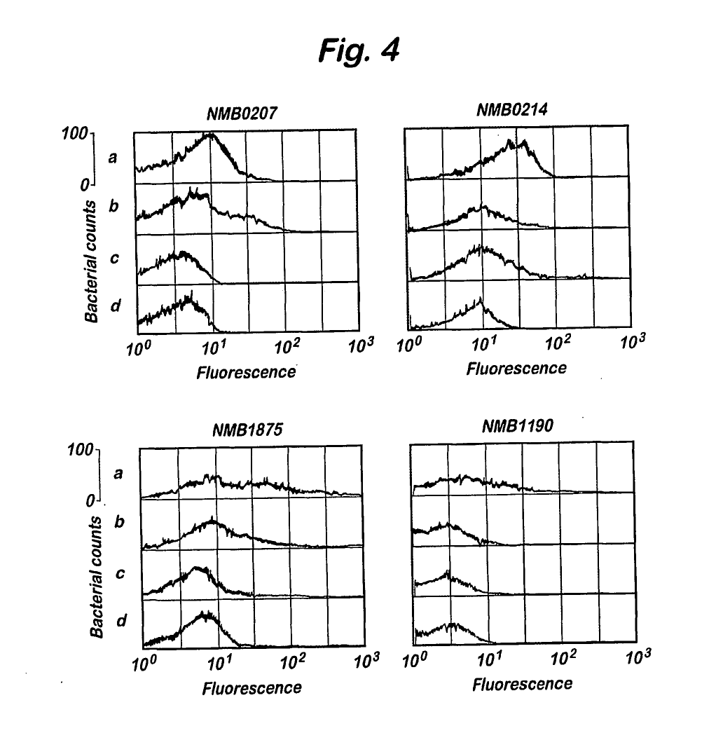

One of the genes up-regulated in both MenB (4.8 fold) and N. lactamica (2.7 fold) is gapA-1 (NMB0207), the gene coding for the metabolic enzyme glyceraldehyde 3-phosphate dehydrogenase (GAPDH). The normal function of GAPDH in cellular metabolism is the conversion of glyceraldehyde 3-phosphate to 1,3-diphosphoglycerate with the concomitant production of NADH. However, in some Gram positive bacteria, the enzyme is exported to the bacterial surface. In Streptococcus pyogenes, GAPDH represents a major surface exposed protein and acts as an ADP-ribosylating enzyme [Lottenberg et al. (1992) J. Bacteriol. 174, 5204-5210; De Matteis et al. (1994) Proc. Natl. Acad. Sci. U.S.A. 91, 1114-1118]. In Streptococcus pneumoniae, the enzyme may be directly involved in the active efflux mechanism of erythromycin [Cash et al. (1999) Electrophoresis 20, 2259-2268]. Furthermore, the enzyme plays an important role in cellular communication by activating host protein phosphorylation mechanisms [Pancholi & Fischetti (1997) J. Exp. Med. 186, 1633-1643]. Finally, in Staphylococcus, the cell-surface-associated GAPDH serves as a surface receptor for transferrin and binds different human serum proteins [Winram & Lottemberg (1996) Microbiology 142, 2311-2320]. In MenB, the presence of two GAPDH genes in the chromosome and the up-regulation of one of these following cell contact suggest a special role for GAPDH. This role was confirmed by FACS analysis which showed that, following cell contact, GAPDH is exported to and accumulated on the bacterial surface (FIG. 4a). This is the first time that GAPDH has been found on the surface of a Gram negative bacterium.

Other Genes

Several further genes belonging to different categories respond to cell contact. For instance, the catalase gene (kat (NMB0216)) was found up-regulated in both bacteria. This is consistent with the fact that producing oxygen radicals [Klebanoff et al. (1983) Ciba Found Symp. 99, 92-112; Ramarao et al. (2000) Mol. Microbiol. 38, 103-113] is one of the mechanisms by which eukaryotic cells try to protect themselves against pathogen aggression.

Genes involved in DNA metabolism are often critical for bacterial pathogenesis and, as for DNA restriction-modification genes, are often located within pathogenicity islands [Salama et al. (2000) Proc. Natl. Acid. Sci. 97:14668-14673] or subjected to phase variation [Ge & Taylo (1999) Annu. Rev. Microbiol. 53:353-387; Saunders et al. (2000) Mol. Microbiol. 37:207-215; Braaten et al. (1994) Cell 76:577-588]. In S. typhimurium, adenine methylation influences the expression of several virulence genes [Heithoff et al. (1999) Science 284:967-970; Garcia-Del Portillo et al. (1999) Proc. Natl. Acad. Sci. U.S.A. 96:11578-11583]. The Neisseria data show that two restriction modification genes (mod (NMB1261), NMB01375), both encoding DNA methylases and genes coding for nucleases, transposases, helicases and ligases (NMB0090, recQ (NMB0274), ligA-1 (NMB0666), NMB1251, gcr (NMB1278), and NMB1798) were up-regulated during adhesion in both MenB and N. lactamica. In addition to these genes, in MenB interaction with epithelial cells promotes transcription of 3 other DNA metabolism genes (xseB (NMB0262), NMB1510 and mutS (NMB2160)) and 3 additional transposase genes (NMB1050 NMB1601, NMB1770).

Proteases, chaperonins and proteins involved in protein stabilization, classified as “protein fate” genes, also contribute to the virulence of several pathogens. Five genes of this class are up-regulated in both Neisseria species (prlC (NMB0214), NMB1428, secY (NMB0162), dnaK (NMB0554), hscB (NMB1383)). Eleven “protein fate” genes are MenB-specific and, among these, the only one to be up-regulated is the dsbA gene (NMB0278) encoding a periplasmic thiol:disulphide oxidoreductase. In E. coli, DsbA plays a role in adhesion by stabilizing type IV fimbriae [Zhang & Donnenberg (1996) Mol. Microbiol. 21:787-797] and in Shigella flexneri it contributes to intracellular survival and propagation [Yu et al. (2000) Infect. Immun. 68:6449-6456].

Sun et al. developed STM to identify MenB virulence genes. Their study identified 73 genes whose inactivation conferred an attenuated phenotype in a mouse model. Nine of the 73 genes were found regulated in this analysis: three genes involved in amino acids synthesis (metF (NMB0943), metH (NMB0944) and gdhA (NMB1710)), the murein transglycosylase B gene (NMB1279), the gene coding for the Cl⁻ channel protein (NMB2006), the translation elongation factor Tu gene (tufA (NMB0139)), down-regulated at 30 minutes of contact with 16HBE14, and three genes of unknown function (NMB0188 and NMB1971, both up-regulated, and NMB1523 that was down-regulated). Four of these nine genes were MenB-specific (metH, tufA, NMB2006 and NMB1523).

Host-cell Contact Induces Surface Remodeling

The microarray data indicated that, following contact with eukaryotic cells, several genes coding for secreted or potentially surface-exposed proteins were up-regulated. In order to find out whether this indeed resulted in a change of the antigenic profile of the bacterium, FACS was used to investigate the appearance of antigens on bacterial surface. FIG. 4 shows an example of this kind of analysis using mouse sera against 4 recombinant proteins, oligopeptidase A (prlc (NMB0214)), GAPDH (gapA-1 (NMB0207)), the alpha component of sulfite reductase (cysJ-2 (NMB1190) and the product of the hypothetical gene NMB1875.

Mouse sera against these four MenB antigens (a and b) and their corresponding pre-immune sera (c and d) were incubated with either epithelial cell-adhering MenB (a and c) or MenB growing in D-MEM (b and d). FACS analysis was performed at 1-hour, 3-hour and 4-hour infection for NMB0207, NMB1875 and NMB0214, and NMB1190, respectively.

The expression of these four proteins on the surface of MenB grown in GC medium was negative by FACS; when the same assay was performed on bacteria grown in the host cell culture medium in the presence of FCS, however, weakly positive signals were detected for GAPDH and NMB1875 (row b), indicating that some FCS components are possibly capable of promoting surface modification in MenB. However, when MenB was allowed to adhere to 16HBE-14 epithelial cells, the induction of all four proteins was clearly detectable.

In further work on surface remodelling, FACS analysis was performed using mouse sera against twelve proteins which showed activated transcription after adhesion (Table VI). The FACS used R-phycoerythrin-conjugated goat F(ab)₂ anti-mouse IgG. As negative controls, FACS analyses of MenB cells with mouse sera against two cytoplasmic proteins are shown (NifU (NMB1380) panel 13, and the ATP-binding protein of amino acid ABC transporter (NMB0789) panel 14). Within these two panels are the Western Blot analyses of MenB total proteins to confirm the expression of the cytoplasmic antigens.

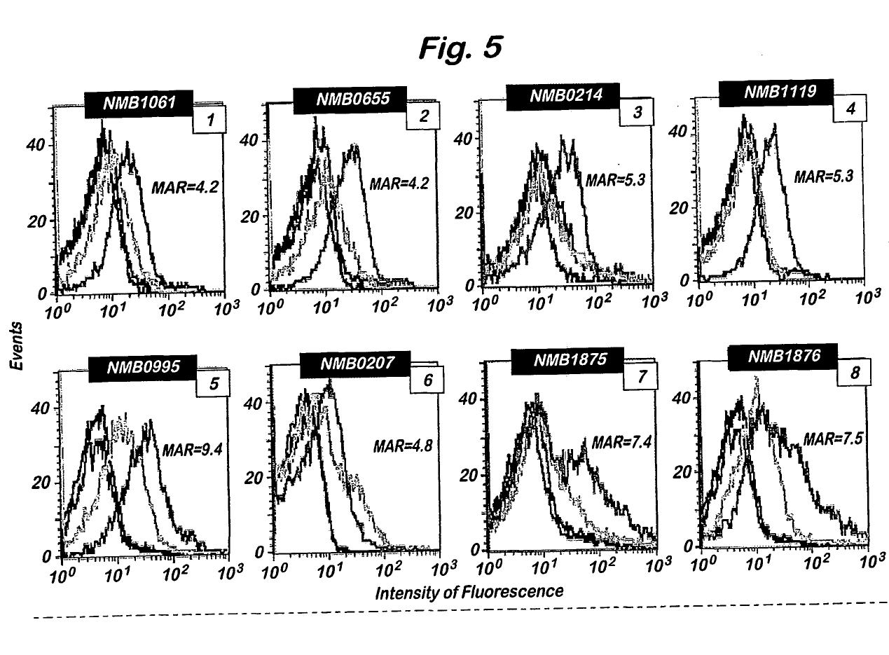

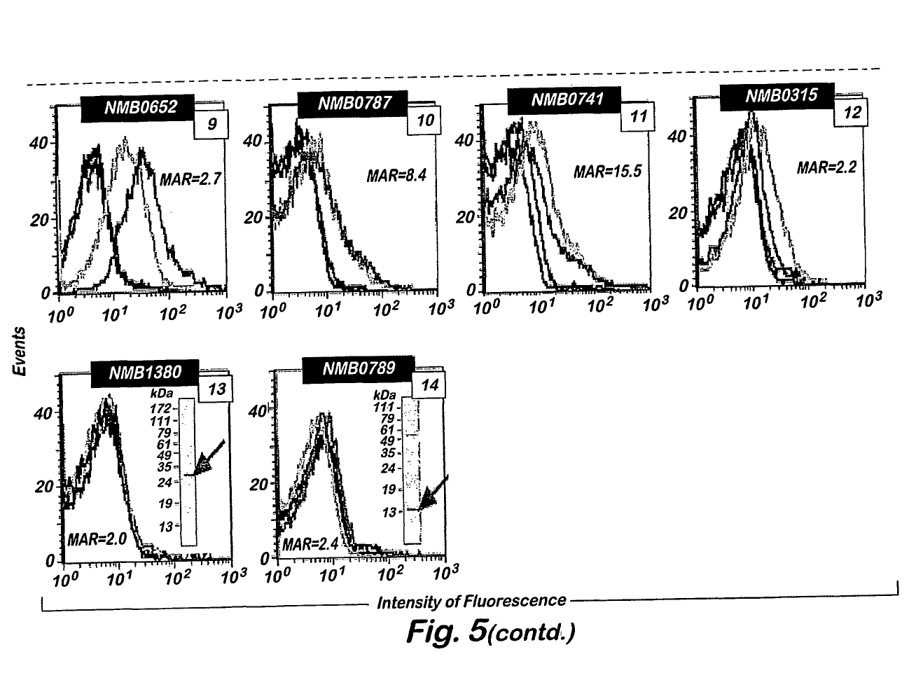

According to computer analysis, six of the twelve activated proteins were peripherally located and six were cytoplasmic. The proteins were selected on the basis of the level and persistence of RNA activation and/or their possible involvement in bacterial adhesion and virulence. As shown in FIG. 5, all proteins were FACS positive. Four of them appeared on the bacterial surface only after adhesion to epithelial cells (panels 1 to 4), 5 proteins were present in non-adhering bacteria but their expression increased upon interaction with the host (panels 5 to 9), 3 proteins were present on the surface of both adhering and non-adhering bacteria and their expression, differently from their corresponding RNA, did not appear to vary upon epithelial cell interaction (panels 10 to 12).

Taken together, these data confirm that interaction with the host involves substantial modification of surface protein components, and that DNA microarrays coupled to FACS analysis with sera against recombinant proteins is an effective approach to identify surface antigens subject to adhesion-dependent modulation.

Serum Bactericidal Activity

The twelve Table VI proteins were tested for the ability of their anti-sera to mediate complement-dependent killing of MenB in a bactericidal assay. Bactericidal activity was evaluated with pooled baby rabbit serum as complement source. Sera against OMV and preimmune sera were used as positive and negative controls, respectively. Titres are expressed as the reciprocal of serum dilution yielding ≧50% bacterial killing as opposed to pre-immune sera.

Of the twelve sera, five showed bactericidal activity against the homologous strain MC58 (Table VII). Two of the bactericidal sera were against hypothetical proteins (the products of NMB0315 and NMB1119 genes) and their function remains to be elucidated. The third bactericidal serum was against the adhesin MafA, one of the two adhesin proteins homologous to gonococcal Maf adhesins. The other two sera were against the MIP-related protein and the enzyme N-acetylglutamate synthase. MIP has been shown to play a role in the survival of intracellular pathogens once inside the host cells and N-acetylglutamate synthetase is a key enzyme in the biosynthesis of arginine from glutamic acid. The protein is predicted to be localised in the cytoplasm, so its presence on the bacterial surface was surprising. Similarly to the findings for GAPDH, this enzyme may function in the metabolism of pathogenic bacteria in a way not yet described.

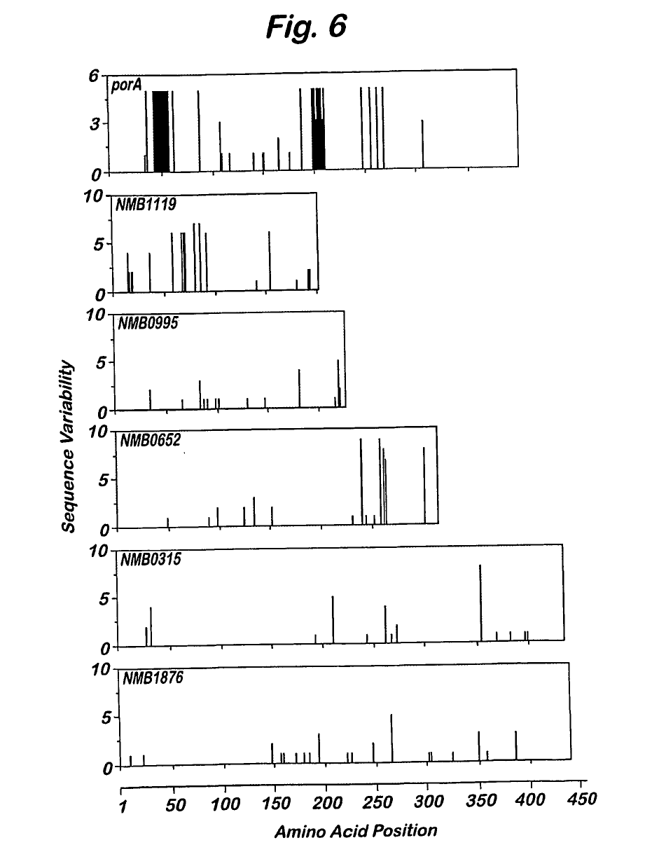

Proteins having specific functions in host-pathogen interaction are likely to be less prone to gene variability. This is a particularly important aspect for MenB whose propensity to sequence variation has historically prevented protein-based vaccines from being developed. To test whether the five bactericidal antigens were conserved, their predicted protein sequences within 11 isolates representative of MenB population and including the four major hypervirulent lineages (ET-5, ET-37, lineage 3, A4) were compared. As shown in FIG. 6, with the exception of NMB1119 (93% conserved), the antigens were highly conserved, ranging from 98 to 99%. Furthermore, and differently from what observed in porA, the amino acid variations were not clustered but rather evenly distributed along the entire protein sequence. The observed sequence conservation was sufficient to allow cross-protection when three of the five sera were tested for bactericidal activity against the heterologous strain 2996 (Table VII).

It will be understood that the invention has been described by way of example only and modifications may be made whilst remaining within the scope and spirit of the invention.

TABLE I
Up-regulated genes

| | | | | | | | | | |
|---|---|---|---|---|---|---|---|---|---|
| NMB0077 | NMB0100 | NMB0366 | NMB0508 | NMB0523 | NMB0541 | NMB0715 | NMB0813 | NMB0928 | NMB1003 |
| NMB1004 | NMB1013 | NMB1048 | NMB1082 | NMB1087 | NMB1108 | NMB1187 | NMB1198 | NMB1370 | NMB1431 |
| NMB1693 | NMB1021 | NMB0760 | NMB0944 | NMB1579 | NMB1582 | NMB1583 | NMB1194 | NMB0527 | NMB1282 |
| NMB1640 | NMB1297 | NMB0977 | NMB1603 | NMB1857 | NMB1799 | NMB1153 | NMB1155 | NMB1189 | NMB1190 |
| NMB1192 | NMB0262 | NMB1510 | NMB2160 | NMB1676 | NMB1845 | NMB0010 | NMB1377 | NMB1830 | NMB0436 |
| NMB1030 | NMB1627 | NMB1665 | NMB1050 | NMB1601 | NMB1770 | NMB0278 | NMB0164 | NMB0875 | NMB1007 |
| NMB1585 | NMB0617 | NMB0689 | NMB0787 | NMB0788 | NMB0789 | NMB0879 | NMB0880 | NMB1017 | NMB0615 |
| NMB0634 | NMB1719 | NMB0204 | NMB0375 | NMB1380 | NMB1448 | NMB1754 | NMB1924 | NMB2006 | NMB0233 |
| NMB0235 | NMB0305 | NMB0306 | NMB0311 | NMB0320 | NMB0328 | NMB0362 | NMB0363 | NMB0489 | NMB0504 |
| NMB0510 | NMB0511 | NMB0517 | NMB0518 | NMB0655 | NMB0902 | NMB0934 | NMB0945 | NMB0965 | NMB0968 |
| NMB1001 | NMB1043 | NMB1148 | NMB1167 | NMB1205 | NMB1215 | NMB1255 | NMB1292 | NMB1369 | NMB1769 |
| NMB1795 | NMB1825 | NMB1875 | NMB0203 | NMB0943 | NMB0637 | NMB1068 | NMB1710 | NMB1876 | NMB0763 |
| NMB0665 | NMB0186 | NMB1379 | NMB0396 | NMB1364 | NMB2069 | NMB0062 | NMB0063 | NMB0178 | NMB1279 |
| NMB1818 | NMB1273 | NMB1533 | NMB0170 | NMB0191 | NMB0216 | NMB0018 | NMB0493 | NMB0001 | NMB0274 |
| NMB0666 | NMB1278 | NMB1261 | NMB1375 | NMB0176 | NMB0206 | NMB0208 | NMB0993 | NMB1803 | NMB0089 |
| NMB0207 | NMB1276 | NMB0050 | NMB0188 | NMB0291 | NMB0292 | NMB0315 | NMB0316 | NMB0455 | NMB0741 |
| NMB1061 | NMB1119 | NMB1128 | NMB1336 | NMB1816 | NMB1828 | NMB0090 | NMB1251 | NMB1798 | NMB0214 |
| NMB1428 | NMB0162 | NMB1383 | NMB0111 | NMB1506 | NMB1595 | NMB0697 | NMB0670 | NMB1252 | NMB1561 |
| NMB1711 | NMB1856 | NMB0646 | NMB0133 | NMB0217 | NMB0177 | NMB0881 | NMB0461 | NMB1990 | NMB0041 |
| NMB0098 | NMB0490 | NMB0652 | NMB0994 | NMB0995 | NMB2016 | | | | |

NB: seven of these genes are up-regulated at one stage during adhesion and down-regulated at a different stage.

TABLE II
Down-regulated genes

| | | | | | | | | | |
|---|---|---|---|---|---|---|---|---|---|
| NMB0102 | NMB0129 | NMB0256 | NMB0260 | NMB0383 | NMB0651 | NMB0657 | NMB0659 | NMB0667 | NMB0754 |
| NMB0979 | NMB1343 | NMB1468 | NMB1490 | NMB1523 | NMB1722 | NMB1848 | NMB1942 | NMB2074 | NMB2137 |
| NMB2141 | NMB0335 | NMB0603 | NMB0801 | NMB1864 | NMB0413 | NMB0420 | NMB0422 | NMB0423 | NMB0382 |
| NMB0171 | NMB0421 | NMB1811 | NMB2095 | NMB0740 | NMB0782 | NMB1322 | NMB1933 | NMB1934 | NMB1940 |
| NMB0251 | NMB1366 | NMB0546 | NMB0671 | NMB0015 | NMB1344 | NMB0390 | NMB0391 | NMB0956 | NMB0959 |
| NMB0960 | NMB1916 | NMB0386 | NMB0419 | NMB0479 | NMB0739 | NMB0771 | NMB0883 | NMB1121 | NMB1494 |
| NMB1312 | NMB1372 | NMB1313 | NMB0059 | NMB0550 | NMB0791 | NMB1519 | NMB1522 | NMB1972 | NMB1973 |
| NMB1302 | NMB0156 | NMB0166 | NMB0609 | NMB0722 | NMB0723 | NMB0876 | NMB0942 | NMB1323 | NMB2101 |
| NMB0124 | NMB0139 | NMB0937 | NMB2102 | NMB0823 | NMB1307 | NMB0815 | NMB0824 | NMB1874 | NMB0380 |
| NMB2075 | NMB0683 | NMB0712 | NMB1509 | NMB0378 | NMB0535 | NMB1207 | NMB0393 | NMB1199 | NMB1220 |
| NMB1381 | NMB0057 | NMB0298 | NMB1211 | NMB0838 | NMB0427 | NMB1305 | NMB1935 | NMB1936 | NMB1937 |
| NMB1938 | NMB1723 | NMB2051 | NMB0947 | NMB1918 | NMB0411 | NMB0428 | NMB1971 | NMB0495 | NMB0700 |
| NMB1802 | NMB0554 | NMB0130 | NMB0131 | NMB0140 | NMB0149 | NMB0153 | NMB0154 | NMB0155 | NMB0157 |
| NMB0158 | NMB0159 | NMB0160 | NMB0161 | NMB0165 | NMB0167 | NMB0169 | NMB0321 | NMB0322 | NMB1320 |
| NMB2056 | NMB2057 | NMB0138 | NMB0168 | NMB0462 | NMB0610 | NMB1794 | NMB1206 | NMB1919 | NMB1657 |
| NMB2017 | | | | | | | | | |

TABLE III
180 meningococcus-specific regulated proteins

| | | | | | Expression ratio at time | | | |
|---|---|---|---|---|---|---|---|---|
| | Gene | | | | (mins) | | | |
| NMB | name | Family | Subfamily | PRODUCT | 30 | 60 | 120 | 180 |
| NMB0077 | | | | | 3.19 | | | |
| NMB0100 | | | | hypothetical protein | 4.28 | | | |
| NMB0102 | | | | hypothetical protein | | 0.49 | | |
| NMB0129 | | | | hypothetical protein | | 0.45 | | |
| NMB0256 | | | | hypothetical protein | | 0.48 | | |
| NMB0260 | | | | hypothetical protein | | 0.44 | | |
| NMB0366 | | | | hypothetical protein | 2.08 | | | |
| NMB0383 | | | | hypothetical protein | 0.20 | | | |
| NMB0508 | | | | hypothetical protein | 4.62 | | | |
| NMB0523 | | | | | | | | 2.51 |
| NMB0541 | | | | hypothetical protein | 3.27 | | | |
| NMB0651 | | | | hypothetical protein | 0.49 | | | |
| NMB0657 | | | | hypothetical protein | 0.10 | 0.45 | | |
| NMB0659 | | | | hypothetical protein | | 0.49 | | |
| NMB0667 | | | | hypothetical protein | 0.47 | | | |
| NMB0715 | | | | hypothetical protein | 2.78 | | | |
| NMB0754 | | | | hypothetical protein | 0.32 | | | |
| NMB0813 | | | | hypothetical protein | 2.14 | | | |
| NMB0928 | | | | hypothetical protein | | 2.06 | | |
| NMB0979 | | | | hypothetical protein | 0.43 | | | |
| NMB1003 | | | | hypothetical protein | 2.81 | | | |
| NMB1004 | | | | hypothetical protein | 3.61 | 2.18 | | |
| NMB1013 | | | | hypothetical protein | 4.71 | | | |
| NMB1048 | | | | hypothetical protein | | | | 2.03 |
| NMB1082 | | | | hypothetical protein | 3.33 | | | |
| NMB1087 | | | | hypothetical protein | | 3.14 | | |
| NMB1108 | | | | hypothetical protein | 2.44 | | 0.47 | |
| NMB1187 | | | | hypothetical protein | 2.63 | | | |
| NMB1198 | | | | | 2.70 | | | |
| NMB1343 | | | | hypothetical protein | 0.40 | | | |
| NMB1370 | | | | hypothetical protein | | 2.05 | | |
| NMB1431 | | | | hypothetical protein | 2.87 | | | |
| NMB1468 | | | | hypothetical protein | 0.36 | 0.48 | | |
| NMB1490 | | | | hypothetical protein | 0.48 | | | |
| NMB1523 | | | | hypothetical protein | 0.36 | | | |
| NMB1693 | | | | hypothetical protein | | 6.25 | | |
| NMB1722 | | | | | 0.44 | | | |
| NMB1848 | | | | hypothetical protein | | 0.47 | | |
| NMB1942 | | | | hypothetical protein | | 0.49 | | |
| NMB2074 | | | | hypothetical protein | 0.40 | | | |
| NMB2137 | | | | hypothetical protein | 0.38 | 0.36 | | 0.48 |
| NMB2141 | | | | hypothetical protein | 0.46 | | | |
| NMB1021 | trpE | Amino acid | Aromatic amino acid family | anthranilate synthase component I | 2.43 | 2.27 | | |
| NMB0335 | dapD | biosynthesis | Aspartate family | 2,3,4,5-tetrahydropyridine-2-carboxylate N- | 0.47 | 0.41 | | |
| | | | | succinyltransferase | | | | |
| NMB0760 | dapF | | Aspartate family | diaminopimelate epimerase | | | 7.52 | |
| NMB0944 | metH | | Aspartate family | 5-methyltetrahydropteroyltriglutamate- | | 2.43 | | |
| | | | | homocysteine methyltransferase | | | | |
| NMB0603 | hisE | | Histidine family | phosphoribosyl-ATP cyclohydrolase | 0.46 | | | |
| NMB1579 | hisG | | Histidine family | ATP phosphoribosyltransferase | 50.25 | | | |
| NMB1582 | hisC | | Histidine family | histidinol-phosphate aminotransferase | | 2.14 | | |
| NMB1583 | hisB | | Histidine family | imidazoleglycerol-phosphate dehydratase | | 2.10 | | |
| NMB0801 | hemB | Biosynthesis of | Heme, porphyrin, and | delta-aminolevulinic acid dehydratase | 0.49 | | | |
| | | cofactors, prosthetic | cobalamin | | | | | |
| NMB1194 | cysG-2 | groups, and carriers | Heme, porphyrin, and | siroheme synthase | | | 4.72 | 3.80 |
| | | | cobalamin | | | | | |
| NMB1864 | hemL | | Heme, porphyrin, and | glutamate-1-semialdehyde 2,1-aminomutase | 0.43 | | | |
| | | | cobalamin | | | | | |
| NMB0527 | | | Other | 6-pyruvoyl tetrahydrobiopterin synthase, putative | 2.32 | | | |
| NMB1282 | panD | | Pantothenate and | aspartate 1-decarboxylase | | 2.06 | | |
| | | | coenzyme A | | | | | |
| NMB1640 | serC | | Pyridoxine | phosphoserine aminotransferase | | | 2.41 | |
| NMB1297 | | Cell envelope | Biosynthesis and | membrane-bound lytic murein transglycosylase D | | 2.03 | | |
| | | | degradation of surface | | | | | |
| | | | polysaccharides and | | | | | |
| | | | lipopolysaccharides | | | | | |
| NMB0413 | penA | | Biosynthesis of murein | penicillin-binding protein 2 | 0.39 | | | |
| | | | sacculus and peptidoglycan | | | | | |
| NMB0420 | murD | | Biosynthesis of murein | UDP-N-acetylmuramoylalanine--D-glutamate | | 0.45 | | |
| | | | sacculus and peptidoglycan | ligase | | | | |
| NMB0422 | murG | | Biosynthesis of murein | UDP-N-acetylglucosamine--N-acetylmuramyl- | 0.42 | 0.38 | | |
| | | | sacculus and peptidoglycan | (pentapeptide) pyrophosphoryl-undecaprenol N- | | | | |
| | | | | acetylglucosamine transferase | | | | |
| NMB0423 | murC | | Biosynthesis of murein | UDP-N-acetylmuramate--alanine ligase | 0.43 | | | |
| | | | sacculus and peptidoglycan | | | | | |
| NMB0382 | rmpM | | Other | outer membrane protein class 4 | 0.21 | | | |
| NMB0171 | minD | Cellular processes | Cell division | septum site-determining protein MinD | 0.48 | | | |
| NMB0421 | ftsW | | Cell division | cell division protein FtsW | 0.49 | 0.46 | | |
| NMB1811 | pilP | | Pathogenesis | pilP protein | 0.48 | | | 0.49 |
| NMB2095 | | | Pathogenesis | adhesin complex protein, putative | 0.21 | | | |
| NMB0977 | | | Toxin production and | modulator of drug activity B, putative | | 3.48 | | |
| | | | resistance | | | | | |
| NMB1603 | | | Toxin production and | tellurite resistance protein, putative | 2.42 | | | |
| | | | resistance | | | | | |
| NMB1857 | mdaB | | Toxin production and | modulator of drug activity B | | 3.91 | | |
| | | | resistance | | | | | |
| NMB1799 | metK | Central intermediary | Other | S-adenosylmethionine synthetase | | | 2.38 | |
| NMB1153 | cysN-1 | metabolism | Sulfur metabolism | sulfate adenylyltransferase, subunit 1 | | 2.26 | 7.51 | 4.73 |
| NMB1155 | cysH-1 | | Sulfur metabolism | phosphoadenosine phosphosulfate reductase | | | 3.22 | 3.24 |
| NMB1189 | cysI-2 | | Sulfur metabolism | sulfite reductase hemoprotein, beta-component | | | 6.64 | 4.01 |
| NMB1190 | cysJ-2 | | Sulfur metabolism | sulfite reductase (NADPH) flavoprotein, alpha | | 2.37 | 6.75 | 3.99 |
| | | | | component | | | | |
| NMB1192 | cysD-2 | | Sulfur metabolism | sulfate adenylyltransferase, subunit 2 | | | 5.00 | 3.27 |
| NMB0262 | xseB | DNA metabolism | Degradation of DNA | exodeoxyribonuclease, small subunit | 3.41 | | | |
| NMB1510 | | | Degradation of DNA | thermonuclease family protein | 2.74 | | | |
| NMB0740 | recN | | DNA replication, | DNA repair protein RecN | 0.48 | | | |
| | | | recombination, and repair | | | | | |
| NMB0782 | radA | | DNA replication, | DNA repair protein RadA | | 0.46 | | |
| | | | recombination, and repair | | | | | |
| NMB1322 | | | DNA replication, | primosomal replication protein n, putative | 0.47 | | | |
| | | | recombination, and repair | | | | | |
| NMB2160 | mutS | | DNA replication, | DNA mismatch repair protein MutS | 2.30 | | | |
| | | | recombination, and repair | | | | | |
| NMB1676 | gcvP | Energy metabolism | Amino acids and amines | | | 2.15 | | |
| NMB1933 | atpC | | ATP-proton motive force | ATP synthase F1, epsilon subunit | | 0.28 | | |
| | | | interconversion | | | | | |
| NMB1934 | atpD | | ATP-proton motive force | ATP synthase F1, beta subunit | | | | 0.49 |
| | | | interconversion | | | | | |
| NMB1940 | atpB | | ATP-proton motive force | ATP synthase F0, A subunit | 0.46 | | | |
| | | | interconversion | | | | | |
| NMB0251 | nuol | | Electron transport | NADH dehydrogenase I, I subunit | | 0.39 | | 0.49 |
| NMB1366 | | | Electron transport | thioredoxin | 0.27 | | | |
| NMB1845 | | | Electron transport | thioredoxin | 2.06 | | | |
| NMB0546 | adhP | | Fermentation | alcohol dehydrogenase, propanol-preferring | 0.42 | | | |
| NMB0010 | pgk | | Glycolysis/gluconeogenesis | phosphoglycerate kinase | 3.23 | | | |
| NMB1377 | lldD | | Glycolysis/gluconeogenesis | L-lactate dehydrogenase | | | 2.08 | |
| NMB0671 | sfcA | | Other | malate oxidoreductase (NAD) | 0.47 | | | |
| NMB0015 | gnd | | Pentose phosphate pathway | 6-phosphogluconate dehydrogenase, | 0.05 | | | |
| | | | | decarboxylating | | | | |
| NMB1344 | lpdA | | Pyruvate dehydrogenase | pyruvate dehydrogenase, E3 component, | 0.37 | | | |
| | | | | lipoamide dehydrogenase | | | | |
| NMB0390 | mapA | | Sugars | maltose phosphorylase | | | | 0.47 |
| NMB0391 | pgmB | | Sugars | beta-phosphoglucomulase | | | | 0.37 |
| NMB1830 | | | Sugars | phosphoglycolate phosphatase, putative | 2.09 | | | |
| NMB0956 | sucB | | TCA cycle | 2-oxoglutarate dehydrogenase, E2 component, | 0.37 | | | |
| | | | | dihydrolipoamide succinyltransferase | | | | |
| NMB0959 | sucC | | TCA cycle | succinyl-CoA synthetase, beta subunit | 0.42 | | | |
| NMB0960 | sucD | | TCA cycle | succinyl-CoA synthetase, alpha subunit | 0.41 | | | |
| NMB1916 | fabH | Fatty acid and | Biosynthesis | 3-oxoacyl-(acyl-carrier-protein) synthase III | 0.45 | | | |
| NMB0386 | pgpA | phospholipid | Degradation | phosphatidylglycerophosphatase A | 0.45 | | | |
| | | metabolism | | | | | | |
| NMB0419 | | Hypothetical | Conserved | conserved hypothetical protein | | 0.36 | | |
| NMB0436 | | proteins | Conserved | conserved hypothetical protein | 2.44 | | | |
| NMB0479 | | | Conserved | conserved hypothetical protein | 0.45 | | | |
| NMB0739 | | | Conserved | conserved hypothetical protein | | 0.23 | | |
| NMB0771 | | | Conserved | conserved hypothetical protein | 0.39 | | | |
| NMB0883 | | | Conserved | conserved hypothetical protein | 0.38 | | | |
| NMB1030 | | | Conserved | conserved hypothetical protein | | 2.25 | | |
| NMB1121 | | | Conserved | | | 0.45 | | |
| NMB1494 | | | Conserved | conserved hypothetical protein | | 0.50 | | |
| NMB1627 | | | Conserved | conserved hypothetical protein | 2.94 | | | |
| NMB1665 | | | Conserved | conserved hypothetical protein | 3.43 | | | |
| NMB1050 | | Other categories | Transposon functions | transposase, IS30 family | 2.17 | | | |
| NMB1601 | | | Transposon functions | IS1106 transposase | 2.11 | | | |
| NMB1770 | | | Transposon functions | transposase, IS30 family | 2.17 | | | |
| NMB1312 | clpP | Protein fate | Degradation of proteins, | ATP-dependent Clp protease, proteolytic subunit | 0.39 | | | |
| | | | peptides, and glycopeptides | | | | | |
| NMB1372 | clpX | | Degradation of proteins, | ATP-dependent Clp protease, ATP-binding | 0.43 | | | |
| | | | peptides, and glycopeptides | subunit ClpX | | | | |
| NMB1313 | tig | | Protein and peptide | trigger factor | 0.48 | | | |
| | | | secretion and trafficking | | | | | |
| NMB0059 | dnaJ | | Protein folding and | dnaJ protein | | 0.43 | | |
| | | | stabilization | | | | | |
| NMB0278 | dsbA-1 | | Protein folding and | thiol: disulfide interchange protein DsbA | | 2.10 | | |
| | | | stabilization | | | | | |
| NMB0550 | dsbC | | Protein folding and | thiol: disulfide interchange protein DsbC | 0.49 | | | |
| | | | stabilization | | | | | |
| NMB0791 | | | Protein folding and | peptidyl-prolyl cis-trans isomerase | 0.36 | | | |
| | | | stabilization | | | | | |
| NMB1519 | dsbD | | Protein folding and | thiol: disulfide interchange protein DsbD | 0.34 | 0.44 | | |
| | | | stabilization | | | | | |
| NMB1522 | slyD | | Protein folding and | FKBP-type peptidyl-prolyl cis-trans isomerase | | 0.28 | | |
| | | | stabilization | SlyD | | | | |
| NMB1972 | groEL | | Protein folding and | chaperonin, 60 kDa | 0.26 | | | |
| | | | stabilization | | | | | |
| NMB1973 | groES | | Protein folding and | chaperonin, 10 kDa | 0.33 | | | |
| | | | stabilization | | | | | |
| NMB1302 | himD | Protein synthesis | Nucleoproteins | integration host factor, beta subunit | 0.16 | 0.35 | | |
| NMB0156 | rpsH | | Ribosomal proteins: | 30S ribosomal protein S8 | 0.37 | | | |
| | | | synthesis and modification | | | | | |
| NMB0164 | rpmJ | | Ribosomal proteins: | 50S ribosomal protein L36 | 2.48 | 0.46 | 3.51 | 7.54 |
| | | | synthesis and modification | | | | | |
| NMB0166 | rpsK | | Ribosomal proteins: | 30S ribosomal protein S11 | 0.38 | | | |
| | | | synthesis and modification | | | | | |
| NMB0609 | rpsO | | Ribosomal proteins: | 30s ribosomal protein S15 | 0.43 | | | |
| | | | synthesis and modification | | | | | |
| NMB0722 | rpml | | Ribosomal proteins: | 50S ribosomal protein L35 | 0.26 | | | |
| | | | synthesis and modification | | | | | |
| NMB0723 | rplT | | Ribosomal proteins: | 50S ribosomal protein L20 | 0.22 | | | |
| | | | synthesis and modification | | | | | |
| NMB0876 | rplY | | Ribosomal proteins: | 50S ribosomal protein L25 | 0.32 | | | |
| | | | synthesis and modification | | | | | |
| NMB0942 | rpmE | | Ribosomal proteins: | 50S ribosomal protein L31, putative | | | | 0.48 |
| | | | synthesis and modification | | | | | |
| NMB1323 | rpsF | | Ribosomal proteins: | 30S ribosomal protein S6 | 0.42 | | | |
| | | | synthesis and modification | | | | | |
| NMB2101 | rpsB | | Ribosomal proteins: | 30S ribosomal protein S2 | 0.27 | | | |
| | | | synthesis and modification | | | | | |
| NMB0124 | tufB | | Translation factors | translation elongation factor Tu | 0.18 | | | |
| NMB0139 | tufA | | Translation factors | translation elongation factor Tu | 0.19 | | | |
| NMB0937 | efp | | Translation factors | elongation factor P (EF-P) | 0.47 | | | |
| NMB2102 | tsf | | Translation factors | elongation factor TS (EF-TS) | 0.13 | | | |
| NMB0823 | adk | Purines, | Nucleotide and nucleoside | adenylate kinase | 0.48 | | | |
| | | pyrimidines, | interconversions | | | | | |
| NMB1307 | ndk | nucleosides, and | Nucleotide and nucleoside | nucleoside diphosphate kinase | 0.35 | | | |
| | | nucleotides | interconversions | | | | | |
| NMB0815 | purA | | Purine ribonucleotide | adenylosuccinate synthetase | 0.45 | | | |
| | | | biosynthesis | | | | | |
| NMB0875 | prsA | | Purine ribonucleotide | ribose-phosphate pyrophosphokinase | 2.26 | 2.51 | | |
| | | | biosynthesis | | | | | |
| NMB0824 | pyrF | | Pyrimidine ribonucleotide | orotidine 5′-phosphate decarboxylase | 0.48 | | | |
| | | | biosynthesis | | | | | |
| NMB1874 | pyrE | | Pyrimidine ribonucleotide | orotate phosphoribosyltransferase | 0.25 | | | |
| | | | biosynthesis | | | | | |
| NMB0380 | | Regulatory | Other | transcriptional regulator, Crp/Fnr family | 0.50 | | | |
| NMB1007 | | functions | Other | transcriptional regulator | 3.10 | | | |
| NMB1585 | | | Other | transcriptional regulator, MarR family | 2.42 | | | |
| NMB2075 | | | Other | BirA protein/Bvg accessory factor | 0.44 | | | |
| NMB0617 | rho | Transcription | Transcription factors | transcription termination factor Rho | 2.33 | 2.84 | | |
| NMB0683 | nusB | | Transcription factors | N utilization substance protein B | 0.47 | | | |
| NMB0689 | greB | | Transcription factors | transcription elongation factor GreB | | | | 6.47 |
| NMB0712 | rpoH | | Transcription factors | RNA polymerase sigma-32 factor | 0.37 | | | |
| NMB0787 | | Transport and | Amino acids, peptides and | amino acid ABC transporter, periplasmic amino | 0.48 | | 8.42 | 2.42 |
| | | binding proteins | amines | acid-binding protein | | | | |
| NMB0788 | | | Amino acids, peptides and | amino acid ABC transporter, permease protein | 0.39 | | 3.56 | |
| | | | amines | | | | | |
| NMB0789 | | | Amino acids, peptides and | amino acid ABC transporter, ATP-binding | | | 2.37 | |
| | | | amines | protein | | | | |
| NMB1509 | | | Amino acids, peptides and | amino acid ABC transporter, permease protein | 0.40 | | | |
| | | | amines | | | | | |
| NMB0378 | | | Anions | phosphate permease, putative | 0.42 | | 0.39 | |
| NMB0879 | cysA | | Anions | sulfate ABC transporter, ATP-binding protein | | | 2.88 | 2.06 |
| NMB0880 | cysW | | Anions | sulfate ABC transporter, permease protein | | | 3.14 | 2.57 |
| NMB1017 | sbp | | Anions | sulfate ABC transporter, periplasmic sulfate- | | 3.24 | 8.10 | 3.00 |
| | | | | binding protein | | | | |
| NMB0535 | gluP | | Carbohydrates, organic | glucose/galactose transporter | 0.41 | | | |
| | | | alcohols, and acids | | | | | |
| NMB0615 | | | Cations | ammonium transporter AmtB, putative | | | 2.27 | |
| NMB0634 | fbpA | | Cations | iron(III) ABC transporter, periplasmic binding | | 2.30 | 2.19 | |
| | | | | protein | | | | |
| NMB1207 | bfrA | | Cations | bacterioferritin A | 0.43 | | | 0.46 |
| NMB0393 | | | Other | multidrug resistance protein | | | 0.48 | |
| NMB1719 | mtrF | | Other | efflux pump component MtrF | | | 4.61 | |
| NMB0204 | smpA | Unknown function | General | lipoprotein, putative | | 2.10 | | |
| NMB0375 | mafA-1 | | General | mafA protein | 2.19 | | | |
| NMB1199 | typA | | General | GTP-binding protein TypA | 0.40 | | | |
| NMB1220 | | | General | stomatin/Mec-2 family protein | 0.45 | | | |
| NMB1380 | | | General | nifU protein | | | 2.01 | |
| NMB1381 | | | General | HesB/YadR/YfhF family protein | 0.47 | | | |
| NMB1448 | dinP | | General | DNA-damage-inducible protein P | 18.09 | 2.14 | | |
| NMB1754 | | | General | cryptic plasmid protein A-related protein | 2.40 | | | |
| NMB1924 | | | General | inositol monophosphatase family protein | 2.04 | | | |
| NMB2006 | | | General | chloride channel protein-related protein | 2.11 | | | |

TABLE IV
167 proteins regulated in both N. meningitidis and N. lactamica

| | | | | | Expression ratio at time (mins) | | | | | | |
|---|---|---|---|---|---|---|---|---|---|---|---|
| | Gene | | | | N. lactamica | | | N. meningitidis | | | |
| NMBID | name | Family | Subfamily | Product | 60 | 120 | 180 | 30 | 60 | 120 | 180 |
| NMB0057 | | | | hypothetical protein | 0.37 | 0.31 | 0.32 | | 0.15 | | |
| NMB0233 | | | | hypothetical protein | 4.75 | | | 2.39 | 4.16 | 2.30 | |
| NMB0235 | | | | hypothetical protein | 10.53 | 2.01 | 2.02 | 2.46 | | | |
| NMB0298 | | | | hypothetical protein | | | 0.45 | 0.33 | | | |
| NMB0305 | | | | hypothetical protein | 2.99 | 2.29 | 2.21 | 4.57 | | | |
| NMB0306 | | | | hypothetical protein | 5.03 | 3.99 | 2.16 | 2.99 | | | |
| NMB0311 | | | | hypothetical protein | 7.44 | 2.03 | 2.52 | 2.15 | | | |
| NMB0320 | | | | hypothetical protein | 3.04 | | 3.78 | 2.45 | | | |
| NMB0328 | | | | hypothetical protein | 3.02 | 2.28 | 2.71 | 2.72 | | | |
| NMB0362 | | | | hypothetical protein | 3.42 | 2.73 | 2.36 | 4.29 | 3.13 | | |
| NMB0363 | | | | hypothetical protein | 2.91 | | | 3.16 | | | |
| NMB0489 | | | | hypothetical protein | 2.43 | | | 2.34 | | | |
| NMB0504 | | | | hypothetical protein | 2.95 | | | 5.38 | 2.00 | | |
| NMB0510 | | | | hypothetical protein | | 2.34 | 2.16 | 2.85 | 2.14 | | |
| NMB0511 | | | | hypothetical protein | 4.20 | 5.07 | | 2.60 | 2.22 | | |
| NMB0517 | | | | hypothetical protein | 2.16 | | | 6.45 | | | |
| NMB0518 | | | | hypothetical protein | 2.01 | | | 2.33 | | | |
| NMB0655 | | | | hypothetical protein | 3.57 | | | 4.23 | 2.79 | | |
| NMB0902 | | | | hypothetical protein | 9.02 | 23.59 | 6.85 | 4.79 | 8.36 | | 3.37 |
| NMB0934 | | | | | 2.59 | 2.42 | | | 2.06 | | |
| NMB0945 | | | | hypothetical protein | 2.14 | | | 213.02 | | | |
| NMB0965 | | | | hypothetical protein | 2.35 | | | | 2.60 | | |
| NMB0968 | | | | hypothetical protein | 3.28 | | | 2.55 | | | |
| NMB1001 | | | | | 4.63 | 6.81 | 4.53 | 4.63 | 4.28 | | |
| NMB1043 | | | | hypothetical protein | 2.34 | | | | 2.40 | | |
| NMB1148 | | | | hypothetical protein | 2.49 | | | 2.04 | | | |
| NMB1167 | | | | hypothetical protein | 9.71 | | 2.03 | 3.10 | | | |
| NMB1205 | | | | hypothetical protein | 3.09 | | | | | 2.45 | |
| NMB1211 | | | | hypothetical protein | | 2.05 | | | 0.47 | | |
| NMB1215 | | | | hypothetical protein | 2.62 | 6.50 | | 3.48 | | | |
| NMB1255 | | | | | 5.88 | 2.13 | | 2.40 | | | |
| NMB1292 | | | | hypothetical protein | | 2.04 | | 3.58 | 2.71 | | |
| NMB1369 | | | | hypothetical protein | | 3.35 | 2.03 | | 2.43 | | |
| NMB1769 | | | | | 3.07 | | | 2.00 | | | |
| NMB1795 | | | | hypothetical protein | 2.85 | 4.11 | 2.52 | 2.05 | 5.77 | | |
| NMB1825 | | | | hypothetical protein | 2.01 | | | 2.03 | | | |
| NMB1875 | | | | hypothetical protein | 2.43 | 3.86 | 2.86 | 7.39 | 5.51 | | |
| NMB0203 | dapB | Amino acid | Aspartate family | dihydrodipicolinate reductase | 5.35 | 2.88 | | 3.22 | 2.40 | | |
| NMB0943 | metF | biosynthesis | Aspartate family | 5,10-methylenetetrahydrofolate | 2.14 | | 2.04 | | 2.48 | | |
| | | | | reductase | | | | | | | |
| NMB0637 | argH | | Glutamate family | argininosuccinate lyase | 3.34 | 3.30 | 3.25 | 2.13 | | | 2.10 |
| NMB1068 | proA | | Glutamate family | gamma-glutamyl phosphate | | 2.19 | | 3.07 | 2.77 | | |
| | | | | reductase | | | | | | | |
| NMB1710 | gdhA | | Glutamate family | glutamate dehydrogenase, | | | 2.20 | | 2.17 | 3.41 | |
| | | | | NADP-specific | | | | | | | |
| NMB1876 | argA | | Glutamate family | N-acetylglutamate synthase | 4.32 | 15.56 | 8.49 | 2.99 | | | 3.54 |
| NMB0763 | cysK | | Serine family | cysteine synthase | | | 2.91 | 0.34 | | 11.46 | 3.88 |
| NMB0665 | | Biosynthesis | Heme, porphyrin, and | oxygen-independent | 8.09 | 17.73 | 24.86 | 13.40 | 32.83 | 3.41 | 5.48 |
| | | of cofactors, | cobalamin | coprophorphyrinogen III oxidase | | | | | | | |
| | | prosthetic | | family protein | | | | | | | |
| NMB0186 | uppS | groups, and | Other | undecaprenyl pyrophosphate | 6.01 | | 2.54 | 2.80 | | | |
| | | carriers | | synthetase | | | | | | | |
| NMB1379 | nifS | | Other | nifS protein | | | 2.10 | | 2.05 | | |
| NMB0396 | nadC | | Pyridine nucleotides | nicotinate-nucleotide | 2.33 | 3.17 | | 3.34 | | | |
| | | | | pyrophosphorylase | | | | | | | |
| NMB1364 | | | Pyridine nucleotides | NH(3)-dependent NAD+ | 2.14 | 2.16 | | 6.04 | | | |
| | | | | synthetase NadE, putative | | | | | | | |
| NMB2069 | thiE | | Thiamine | thiamin-phosphate | 2.04 | 2.41 | | 2.06 | | | |
| | | | | pyrophosphorylase | | | | | | | |
| NMB0062 | rfbA-1 | Cell | Biosynthesis and | glucose-1-phosphate | 5.18 | 3.35 | 2.03 | 3.66 | 2.44 | | |
| | | envelope | degradation of surface | thymidylyltransferase | | | | | | | |
| | | | polysaccharides and | | | | | | | | |
| | | | lipopolysaccharides | | | | | | | | |
| NMB0063 | rfbB-1 | | Biosynthesis and | dTDP-D-glucose | 10.64 | | 3.79 | 2.65 | 5.72 | | |
| | | | degradation of surface | 4,6-dehydratase | | | | | | | |
| | | | polysaccharides and | | | | | | | | |
| | | | lipopolysaccharides | | | | | | | | |
| NMB0178 | lpxA | | Biosynthesis and | acyl-(acyl-carrier-protein)- | 4.14 | 3.37 | | 3.25 | | | |
| | | | degradation of surface | UDP-N-acetylglucosamine | | | | | | | |
| | | | polysaccharides and | O-acyltransferase | | | | | | | |
| | | | lipopolysaccharides | | | | | | | | |
| NMB1279 | | | Biosynthesis and | membrane-bound lytic murein | 5.28 | 2.24 | | 5.57 | 3.71 | | 0.47 |
| | | | degradation of surface | transglycosylase B, putative | | | | | | | |
| | | | polysaccharides and | | | | | | | | |
| | | | lipopolysaccharides | | | | | | | | |
| NMB1818 | | | Biosynthesis and | lipopolysaccharide biosynthesis | 2.04 | 2.25 | | 3.31 | | | |
| | | | degradation of surface | protein, putative | | | | | | | |
| | | | polysaccharides and | | | | | | | | |
| | | | lipopolysaccharides | | | | | | | | |
| NMB1273 | | | Other | alginate O-acetylation protein | | 2.65 | | 2.56 | | | |
| | | | | Algl, putative | | | | | | | |
| NMB1533 | | | Other | H.8 outer membrane protein | | | 0.47 | 0.25 | | | |
| NMB0838 | | Cellular | Adaptations to atypical | cold-shock domain family | | | 0.41 | 0.27 | 0.47 | | |
| | | processes | conditions | protein | | | | | | | |
| NMB0170 | minC | | Cell division | septum site-determining | 4.10 | 2.26 | 2.14 | 4.67 | | | |
| | | | | protein MinC | | | | | | | |
| NMB0191 | | | Cell division | ParA family protein | 2.32 | | | 2.35 | 3.09 | | |
| NMB0427 | ftsZ | | Cell division | cell division protein FtsZ | 0.49 | | | 0.42 | | | |
| NMB0216 | kat | | Detoxification | catalase | 5.58 | 4.53 | | | 2.12 | | |
| NMB0018 | pilE | | Pathogenesis | pilin PilE | 3.79 | 10.40 | 6.89 | 2.84 | | | |
| NMB0493 | | | Pathogenesis | hemagglutinin/hemolysin-related | 2.19 | | | 3.73 | | | |
| | | | | protein | | | | | | | |
| NMB0001 | | Central | Other | acetyltransferase, putative | | 0.42 | | 2.30 | | | |
| NMB1305 | | intermediary | Other | esterase, putative | | 0.36 | 0.46 | 0.45 | | | |
| | | metabolism | | | | | | | | | |
| NMB0274 | recQ | DNA | DNA replication, | ATP-dependent DNA helicase | 2.19 | | | 2.56 | | | |
| | | metabolism | recombination, and repair | RecQ | | | | | | | |
| NMB0666 | ligA-1 | | DNA replication, | DNA ligase | 2.02 | | | 3.07 | | | |
| | | | recombination, and repair | | | | | | | | |
| NMB1278 | gcr | | DNA replication, | site-specific recombinase | 6.62 | 2.56 | | 2.02 | | | |
| | | | recombination, and repair | | | | | | | | |
| NMB1261 | mod | | Restriction/modification | | 5.47 | 2.47 | 2.42 | 2.13 | | | |
| NMB1375 | | | Restriction/modification | | 12.94 | 8.29 | | 24.40 | 2.49 | | |
| NMB0176 | dadA | Energy | Amino acids and amines | D-amino acid dehydrogenase, | 3.41 | 4.20 | 2.03 | 2.60 | 2.37 | | |
| | | metabolism | | small subunit | | | | | | | |
| NMB0206 | aat | | Amino acids and amines | leucyl/phenylalanyl- | 9.60 | | | 2.76 | 2.79 | | |
| | | | | tRNA-protein transferase | | | | | | | |
| NMB1935 | atpG | | ATP-proton motive force | ATP synthase F1, gamma | 0.44 | 0.36 | 0.49 | | 0.45 | | |
| | | | interconversion | subunit | | | | | | | |
| NMB1936 | atpA | | ATP-proton motive force | ATP synthase F1, alpha subunit | 0.42 | 0.36 | 0.47 | 0.36 | | | 0.46 |
| | | | interconversion | | | | | | | | |
| NMB1937 | atpH | | ATP-proton motive force | ATP synthase F1, delta subunit | 0.40 | 0.39 | | 0.50 | | | |
| | | | interconversion | | | | | | | | |
| NMB1938 | atpF | | ATP-proton motive force | ATP synthase F0, B subunit | 0.49 | 0.48 | | 0.41 | 0.47 | | |
| | | | interconversion | | | | | | | | |
| NMB0208 | | | Electron transport | ferredoxin, 4Fe—4S bacterial | 2.52 | 2.88 | | 8.62 | 2.77 | | |
| | | | | type | | | | | | | |
| NMB0993 | | | Electron transport | rubredoxin | | | 3.88 | | | 3.03 | 2.21 |
| NMB1723 | fixP | | Electron transport | cytochrome c oxidase, | 0.46 | | | | | | 0.48 |
| | | | | subunit III | | | | | | | |
| NMB1803 | | | Electron transport | cytochrome c-type biogenesis | | 0.33 | | 2.12 | 0.30 | | |
| | | | | protein, putative | | | | | | | |
| NMB2051 | petC | | Electron transport | ubiquinol-cytochrome c | 0.44 | | | 0.48 | | | |
| | | | | reductase, cytochrome c1 | | | | | | | |
| NMB0089 | pykA | | Glycolysis/gluconeogenesis | pyruvate kinase II | 5.03 | 2.17 | | 2.02 | | | |
| NM80207 | gapA-1 | | Glycolysis/gluconeogenesis | glyceraldehyde 3-phosphate | 2.78 | 2.35 | | 4.82 | | | |
| | | | | dehydrogenase | | | | | | | |
| NMB0947 | | | TCA cycle | lipoamide dehydrogenase, | | 0.37 | | 0.49 | | | |
| | | | | putative | | | | | | | |
| NMB1918 | fabD | Fatty acid | Biosynthesis | malonyl CoA-acyl carrier | 0.42 | 0.35 | 0.47 | 0.29 | | | 0.49 |
| | | and | | protein transacylase | | | | | | | |
| NMB1276 | fadD-1 | phospholipid | Degradation | long-chain-fatty-acid-CoA ligase | 6.54 | 4.34 | 2.34 | 2.05 | | | |
| | | metabolism | | | | | | | | | |
| NMB0050 | | Hypothetical | Conserved | conserved hypothetical protein | 2.28 | 3.11 | 2.26 | 2.55 | | | |
| NMB0188 | | proteins | Conserved | conserved hypothetical protein | 6.31 | | | 2.31 | | | |
| NMB0291 | | | Conserved | conserved hypothetical protein | | | 3.78 | | 2.85 | | 2.85 |
| NMB0292 | | | Conserved | conserved hypothetical protein | | | 3.76 | | 2.36 | | 3.11 |
| NMB0315 | | | Conserved | conserved hypothetical protein | | 3.14 | 2.02 | | 2.18 | | |
| NMB0316 | | | Conserved | conserved hypothetical protein | | 0.47 | | | | 2.03 | |
| NMB0411 | | | Conserved | conserved hypothetical protein | 0.44 | | | 0.31 | 0.47 | | |
| NMB0428 | | | Conserved | conserved hypothetical protein | 0.42 | 0.40 | | 0.45 | | | |
| NMB0455 | | | Conserved | conserved hypothetical protein | 10.61 | 10.00 | 3.57 | 26.41 | 10.78 | | |
| NMB0741 | | | Conserved | conserved hypothetical protein | 9.60 | 10.27 | 4.61 | 15.53 | 9.43 | | 2.16 |
| NMB1061 | | | Conserved | conserved hypothetical protein | 3.71 | 7.36 | 4.18 | 4.20 | 3.26 | | |
| NMB1119 | | | Conserved | conserved hypothetical protein | 4.19 | 5.01 | 2.67 | 5.28 | 4.88 | | |
| NMB1128 | | | Conserved | conserved hypothetical protein | 4.93 | 6.61 | 4.25 | 20.72 | 7.55 | | |
| NMB1336 | | | Conserved | conserved hypothetical protein | 3.17 | 5.13 | 6.79 | 4.41 | 3.41 | | |
| NMB1816 | | | Conserved | conserved hypothetical protein | 2.80 | 4.69 | 2.61 | 2.14 | 2.62 | | |
| NMB1828 | | | Conserved | conserved hypothetical protein | | | 0.47 | 2.83 | | | |
| NMB1971 | | | Conserved | conserved hypothetical protein | 5.76 | 15.19 | 9.55 | 2.98 | | | |
| NMB0495 | | Other | Plasmid functions | replication protein | | 0.42 | | 0.43 | | | |
| NMB0090 | | categories | Transposon functions | | 3.02 | 3.68 | 2.45 | 2.03 | | | |
| NMB1251 | | | Transposon functions | transposase, IS30 family | 7.29 | 3.25 | 2.04 | | 2.68 | | |
| NMB1798 | | | Transposon functions | | 3.90 | | 2.06 | 4.14 | | | |
| NMB0214 | prfC | Protein fate | Degradation of proteins, | oligopeptidase A | 5.00 | 3.08 | 2.07 | 5.27 | 3.65 | | |
| | | | peptides, and glycopeptides | | | | | | | | |
| NMB0700 | iga | | Degradation of proteins, | IgA-specific serine | 0.44 | | | 0.47 | | | |
| | | | peptides, and glycopeptides | endopeptidase | | | | | | | |
| NMB1428 | | | Degradation of proteins, | aminopeptidase, putative | | 2.16 | | 2.56 | 2.30 | | |
| | | | peptides, and glycopeptides | | | | | | | | |
| NMB1802 | gcp | | Degradation of proteins, | O-sialoglycoprotein | | 0.37 | | | 0.36 | | |
| | | | peptides, and glycopeptides | endopeptidase | | | | | | | |
| NMB0162 | secY | | Protein and peptide | preprotein translocase SecY | 5.34 | 3.18 | 2.06 | 2.77 | | | |
| | | | secretion and trafficking | subunit | | | | | | | |
| NMB0554 | dnaK | | Protein folding and | dnaK protein | | 2.05 | 3.15 | 0.32 | | | |
| | | | stabilization | | | | | | | | |
| NMB1383 | hscB | | Protein folding and | chaperone protein HscB | 2.28 | | 2.10 | | 2.10 | | |
| | | | stabilization | | | | | | | | |
| NMB0130 | rplJ | Protein | Ribosomal proteins: | 50S ribosomal protein L10 | | 0.47 | 0.47 | | 0.46 | | |
| | | synthesis | synthesis and modification | | | | | | | | |
| NMB0131 | rplL | | Ribosomal proteins: | 50S ribosomal protein L7/L12 | | | 0.49 | | 0.31 | | |
| | | | synthesis and modification | | | | | | | | |
| NMB0140 | rpsJ | | Ribosomal proteins: | 30S ribosomal protein S10 | | 0.45 | 0.44 | 0.16 | | | |
| | | | synthesis and modification | | | | | | | | |
| NMB0149 | rplP | | Ribosomal proteins: | 50S ribosomal protein L16 | | 0.43 | | | 0.46 | | |
| | | | synthesis and modification | | | | | | | | |
| NMB0153 | rplX | | Ribosomal proteins: | 50S ribosomal protein L24 | | 0.43 | 0.47 | 0.39 | | | |
| | | | synthesis and modification | | | | | | | | |
| NMB0154 | rplE | | Ribosomal proteins: | 50S ribosomal protein L5 | | 2.99 | 2.08 | 0.41 | | | |
| | | | synthesis and modification | | | | | | | | |
| NMB0155 | rpsN | | Ribosomal proteins: | 30S ribosomal protein S14 | | 0.39 | 0.48 | 0.32 | | | |
| | | | synthesis and modification | | | | | | | | |
| NMB0157 | rplF | | Ribosomal proteins: | 50S ribosomal protein L6 | | 0.44 | | 0.42 | | | |
| | | | synthesis and modification | | | | | | | | |
| NMB0158 | rplR | | Ribosomal proteins: | 50S ribosomal protein L18 | | 0.42 | | 0.26 | | | |
| | | | synthesis and modification | | | | | | | | |
| NMB0159 | rpsE | | Ribosomal proteins: | 30s ribosomal protein S5 | | 0.42 | | 0.32 | | | |
| | | | synthesis and modification | | | | | | | | |
| NMB0160 | rpmD | | Ribosomal proteins: | 50S ribosomal protein L30 | 0.47 | 0.37 | | 0.28 | | | |
| | | | synthesis and modification | | | | | | | | |
| NMB0161 | rplO | | Ribosomal proteins: | 50S ribosomal protein L15 | 0.34 | 0.32 | | 0.19 | | | |
| | | | synthesis and modification | | | | | | | | |
| NMB0165 | rpsM | | Ribosomal proteins: | 30S ribosomal protein S13 | | 0.49 | | 0.47 | | | |
| | | | synthesis and modification | | | | | | | | |
| NMB0167 | rpsD | | Ribosomal proteins: | 30S ribosomal protein S4 | | 0.41 | 0.47 | | 0.42 | | |
| | | | synthesis and modification | | | | | | | | |
| NMB0169 | rplQ | | Ribosomal proteins: | 50S ribosomal protein L17 | 0.50 | 0.40 | | 0.31 | 0.49 | | |
| | | | synthesis and modification | | | | | | | | |
| NMB0321 | rpmB | | Ribosomal proteins: | 50S ribosomal protein L28 | | 0.48 | 0.47 | | 0.45 | | |
| | | | synthesis and modification | | | | | | | | |
| NMB0322 | rpmG | | Ribosomal proteins: | 50S ribosomal protein L33 | | 0.45 | 0.37 | 0.46 | 0.19 | | |
| | | | synthesis and modification | | | | | | | | |
| NMB1320 | rplI | | Ribosomal proteins: | 50S ribosomal protein L9 | | 0.45 | 0.48 | 0.23 | | | |
| | | | synthesis and modification | | | | | | | | |
| NMB2056 | rpsI | | Ribosomal proteins: | 30S ribosomal protein S9 | | 0.47 | 0.47 | 0.20 | | | |
| | | | synthesis and modification | | | | | | | | |
| NMB2057 | rplM | | Ribosomal proteins: | 50S ribosomal protein L13 | | 0.44 | 0.38 | 0.25 | | | |
| | | | synthesis and modification | | | | | | | | |
| NMB0138 | fusA | | Translation factors | elongation factor G (EF-G) | | | 0.32 | 0.21 | | | |
| NMB0111 | fmt | | tRNA aminoacylation | methionyl-tRNA | 4.01 | 4.66 | | 3.88 | | | |
| | | | | formyltransferase | | | | | | | |
| NMB1506 | argS | | tRNA aminoacylation | arginyl-tRNA synthetase | 2.60 | 2.25 | | 3.85 | 2.97 | | |
| NMB1595 | alaS | | tRNA aminoacylation | alanyl-tRNA synthetase | | 2.64 | | 7.61 | 3.96 | | |
| NMB0697 | ksgA | | tRNA and rRNA base | dimethyladenosine transferase | 3.64 | | 3.73 | 4.69 | | | |
| | | | modification | | | | | | | | |
| NMB0670 | tmk | Purines, | Nucleotide and nucleoside | thymidylate kinase | | 2.17 | | 2.47 | | | |
| | | pyrimidines, | interconversions | | | | | | | | |
| NMB1252 | purM | nucleosides, | Purine ribonucleotide | phospho- | 7.49 | 2.95 | | | | 2.59 | |
| | | and | biosynthesis | ribosylformylglycinamidine | | | | | | | |
| | | nucleotides | | cyclo-ligase | | | | | | | |
| NMB1561 | | Regulatory | Other | transcriptional regulator, | | 3.85 | 3.03 | 9.54 | 2.79 | | |
| | | functions | | DeoR family | | | | | | | |
| NMB1711 | | | Other | transcriptional regulator, | 2.13 | 2.52 | | 2.33 | 2.52 | | |
| | | | | GntR family | | | | | | | |
| NMB1856 | | | Other | transcriptional regulator, | 2.30 | | | 3.09 | | | |
| | | | | LysR family | | | | | | | |
| NMB0646 | | Transcription | Degradation of RNA | ribonuclease inhibitor barstar | 2.41 | | | | 58.98 | | |
| NMB0133 | rpoC | | DNA-dependent RNA | DNA-directed RNA | 6.93 | | | 2.25 | | | |
| | | | polymerase | polymerase, beta' subunit | | | | | | | |
| NMB0168 | rpoA | | DNA-dependent RNA | DNA-directed RNA polymerase, | | 0.40 | 0.43 | 0.39 | | | |
| | | | polymerase | alpha subunit | | | | | | | |
| NMB0217 | | | Transcription factors | RNA polymerase sigma-54 | 4.18 | 3.83 | | 2.42 | | | |
| | | | | factor RpoN, putative | | | | | | | |
| NMB0177 | | Transport | Amino acids, peptides and | sodium/alanine symporter, | 4.23 | 4.07 | 2.23 | 2.36 | | | |
| | | and binding | amines | putative | | | | | | | |
| NMB0462 | potD-1 | proteins | Amino acids, peptides and | spermidine/putrescine ABC | | 0.42 | 0.34 | | 0.35 | | |
| | | | amines | transporter, periplasmic | | | | | | | |
| | | | | spermidine/putrescine- | | | | | | | |
| | | | | binding protein | | | | | | | |
| NMB0610 | potA-1 | | Amino acids, peptides and | spermidine/putrescine ABC | | | 0.44 | 0.48 | | | |
| | | | amines | transporter, ATP-binding protein | | | | | | | |
| NMB0881 | cysT | | Anions | sulfate ABC transporter, | | 2.16 | | | | 4.97 | 3.15 |
| | | | | permease protein | | | | | | | |
| NMB1794 | | | Carbohydrates, organic | citrate transporter | | 0.50 | | | 0.48 | | |
| | | | alcohols, and acids | | | | | | | | |
| NMB0461 | tbp1 | | Cations | transferrin-binding protein 1 | | 4.58 | 2.60 | | 2.51 | | |
| NMB1206 | bfrB | | Cations | bacterioferritin B | | 0.45 | | 0.40 | | 0.39 | 0.30 |
| NMB1990 | | | Cations | iron(III) ABC transporter, | 2.14 | 2.16 | | 5.78 | 2.69 | | |
| | | | | permease protein | | | | | | | |
| NMB0041 | | | Unknown substrate | ABC transporter, periplasmic | | 2.94 | 2.40 | 9.16 | 3.60 | | 2.12 |
| | | | | solute-binding protein | | | | | | | |
| NMB0098 | | | Unknown substrate | | 3.89 | 3.16 | 2.81 | 4.65 | | | |
| NMB1919 | | | Unknown substrate | ABC transporter, ATP-binding | 0.48 | 0.36 | | 0.47 | 0.44 | | |
| | | | | protein | | | | | | | |
| NMB0490 | | Unknown | General | PspA-related protein | 3.57 | 2.98 | | 2.54 | | | |
| NMB0652 | mafA-2 | function | General | mafA protein | | 2.28 | | 2.71 | | | |
| NMB0994 | | | General | acyl-CoA dehydrogenase family | | | 5.33 | | 2.36 | 8.99 | 3.50 |
| | | | | protein | | | | | | | |
| NMB0995 | | | General | macrophage infectivity | | | 2.67 | | 2.47 | 9.37 | 3.21 |
| | | | | potentiator-related protein | | | | | | | |
| NMB1657 | | | General | comE operon protein 1-related | 0.25 | 0.24 | 0.26 | | 0.09 | | |
| | | | | protein | | | | | | | |
| NMB2016 | | | General | type IV pilin-related protein | | 2.69 | 2.79 | 2.55 | | | |
| NMB2017 | | | General | ComEA-related protein | 2.18 | | | | | 0.48 | |

TABLE V
Most highly up-regulated MenB genes

| | | Adhesion time | | | |
|---|---|---|---|---|---|
| Family | Gene name | 30′ | 60′ | 120′ | 180′ |
| Amino acid biosynthesis | NMB0760 | | | 7.5 | |
| | NMB0763 | 0.3 | | 11.5 | 3.9 |
| | NMB1579 | 50.3 | | | |
| Biosynthesis of | NMB1194-1156 | | | 4.7 | 3.8 |
| cofactors, prosthetic | NMB1364 | 6.0 | | | |
| groups, carriers | NMB0665 | 13.4 | 32.8 | 3.4 | 5.5 |
| Cell envelope | NMB0063 | 2.6 | 5.7 | | |
| | NMB1279 | 5.6 | 3.7 | | 0.5 |
| Cellular processes | NMB0170 | 4.7 | | | |
| Central intermediary | NMB1153-1191 | | 2.3 | 7.5 | 4.7 |
| metabolism | NMB1189-1151 | | | 6.6 | 4.0 |
| | NMB1190-1152 | | 2.4 | 6.8 | 4.0 |
| | NMB1192-1154 | | | 5.0 | 3.3 |
| DNA metabolism | NMB1375 | 24.4 | 2.5 | | |
| Energy metabolism | NMB0207 | 4.8 | | | |
| | NMB0208 | 8.6 | 2.8 | | |
| Other categories | NMB1798 | 4.1 | | | |
| Protein fate | NMB0214 | 5.3 | 3.7 | | |
| Protein synthesis | NMB0164 | 2.5 | 0.5 | 3.5 | 7.5 |
| | NMB0697 | 4.7 | | | |
| | NMB1595 | 7.6 | 4.0 | | |
| Regulatory functions | NMB1561 | 9.5 | 2.8 | | |
| Transcription | NMB0646 | | 59.0 | | |
| | NMB0689 | | | | 6.5 |
| Transport and binding | NMB0041 | 9.2 | 3.6 | | 2.1 |
| proteins | NMB0098 | 4.7 | | | |
| | NMB0787 | 0.5 | | 8.4 | 2.4 |
| | NMB0881 | | | 5.0 | 4.1 |
| | NMB1017 | | 3.2 | 8.1 | 3.0 |
| | NMB1719 | | | 4.6 | |
| | NMB1990 | 5.8 | 2.7 | | |
| Unknown function | NMB0994 | | 2.4 | 9.0 | 3.5 |
| | NMB0995 | | 2.5 | 9.4 | 3.2 |
| | NMB1448 | 18.1 | 2.1 | | |
| Conserved hypothetical | NMB0455 | 26.4 | 10.8 | | |
| and hypothetical proteins | NMB0741 | 15.5 | 9.4 | | 2.2 |
| | NMB1061 | 4.2 | 3.3 | | |
| | NMB1119 | 5.3 | 4.9 | | |
| | NMB1128-1166 | 20.7 | 7.6 | | |
| | NMB1336 | 4.4 | 3.4 | | |
| | NMB0100 | 4.3 | | | |
| | NMB0233 | 2.4 | 4.2 | 2.3 | |
| | NMB0305 | 4.6 | | | |
| | NMB0362 | 4.3 | 3.1 | | |
| | NMB0504 | 5.4 | 2.0 | | |
| | NMB0508 | 4.6 | | | |
| | NMB0517 | 6.4 | | | |
| | NMB0655 | 4.2 | 2.8 | | |
| | NMB0902 | 4.8 | 8.4 | | 3.4 |
| | NMB0945 | 213.0 | | | |
| | NMB1001 | 4.6 | 4.3 | | |
| | NMB1013 | 4.7 | | | |
| | NMB1693 | | 6.3 | | |
| | NMB1795 | 2.1 | 5.8 | | |
| | NMB1875 | 7.4 | 5.5 | | |

TABLE VI
N. meningitidis GENES SELECTED FOR FACS ANALYSIS

| | | | RNA activation | | | | Predicted |
|---|---|---|---|---|---|---|---|
| Gene locus | Family | Product/Function | 30′ | 1 h | 2 h | 3 h | location |
| NMB0207 | Energy metabolism | glyceraldehyde 3-P DHase (gapA-1)/virulence | 4.8 | | | | cytoplasm |
| NMB0214 | Protein fate | oligopeptidase A (prlC)/virulence | 5.3 | 3.7 | | | cytoplasm |
| NMB0315 | Hypothetical proteins | | | 2.2 | | | periplasmic space |
| NMB0655 | Hypothetical proteins | | 4.2 | 2.8 | | | cytoplasm |
| NMB0652 | Unknown | mafA prt (mafA-2)/adhesion | 2.7 | | | | outer membrane |
| NMB0741 | Hypothetical proteins | | 15.5 | 9.4 | | 2.2 | inner membrane |
| NMB0787 | Transport and binding proteins | AA ABC transp, periplasmic AA-binding protein | 0.5 | | 8.4 | 2.4 | outer membrane |
| NMB0995 | Unknown | macrophage infectivity potentiator-related protein | | 2.5 | 9.4 | 3.2 | inner membrane |
| NMB1061 | Hypothetical proteins | | 4.2 | 3.3 | | | cytoplasm |
| NMB1119 | Hypothetical proteins | | 5.3 | 4.9 | | | cytoplasm |
| NMB1875 | Hypothetical proteins | | 7.4 | 5.5 | | | inner membrane |
| NMB1876 | Amino acid biosynthesis | N-acetylglutamate synthetase (argA) | 3.0 | | | 3.5 | cytoplasm |

TABLE VII
BACTERICIDAL ACTIVITY OF TABLE VI PROTEINS

| | | Bactericidal activity | |
|---|---|---|---|
| Gene | Annotation | MC58 | 2996 |
| NMB0315 | Hypothetical prt. | 1/512 | 1/1024 |
| NMB1119 | Hypothetical prt. | 1/384 | 1/1024 |
| NMB0995 | MIP-related prt | 1/750 | n.d. |
| NMB0652 (mafA) | MAFA | 1/1024 | 1/200 |
| NMB1876 (argA) | N-acetylglutamate synthase | 1/1024 | n.d. |

1. A method for preventing the attachment of a Neisserial cell to an epithelial cell, wherein the ability of an adhesion-specific protein to bind to the epithelial cell is blocked.

2. The method of claim 1, wherein the ability to bind is blocked using (i) an antibody specific for the adhesion-specific protein, (ii) an antagonist of the interaction between the adhesion-specific protein and its receptor on the epithelial cell, and/or (iii) a soluble form of the receptor on the epithelial cell.

3. A method for preventing the attachment of a Neisserial cell to an epithelial cell, wherein protein expression from an adhesion-specific gene is inhibited.

4. The method of claim 3, wherein protein expression is inhibited by antisense.

5. A method for preventing the attachment of a Neisseria bacterium to an epithelial cell, wherein one or more adhesion-specific gene (s) in the bacterium is knocked out.

6. A method for preventing the attachment of a Neisserial cell to an epithelial cell, wherein one or more adhesion-specific gene (s) has a mutation which inhibits its activity.

7. A method for determining whether a Neisseria bacterium of interest is in the species meningitides, comprising the step(s) of: (a) contacting the bacterium with a nucleic acid probe comprising the sequence of a MenB-specific adhesion-specific gene or a fragment thereof; and/or (b) contacting the bacterium with an antibody which binds to a MenB-specific adhesion-specific protein or an epitope thereof.

8. The method of claim 7, comprising the further step of detecting the presence or absence of an interaction between the bacterium of interest and the MenB-specific nucleic acid or protein.

9. The method of claim 7 or claim 8, wherein the method confirms that the bacterium of interest is not Neisseria lactamica.

10. A method for identifying a compound that inhibits the binding of a Neisserial cell to an epithelial cell, wherein an adhesion-specific protein is incubated with the epithelial cell and a test compound.

11. The method of claim 10 wherein the test compound is selected from the group consisting of small organic molecules, peptides, peptoids, polypeptides, lipids, metals, nucleotides, nucleosides, polyamines, antibodies, and derivatives thereof.

12. A compound identified by the method of claim 10.

13. A nucleic acid array comprising at least 100 adhesion-specific nucleic acid sequences, or fragments thereof.

14. An antibody which is specific for an adhesion-specific protein.

15. The antibody of claim 14, having an affinity for the adhesion-specific protein of at least 10⁻⁷ M.

16. A nucleic acid comprising a fragment of 8 or more nucleotides from one or more adhesion-specific genes.

17. The nucleic acid of claim 16, wherein the nucleic acid is single-stranded.

18. A nucleic acid of the formula 5′-(N)ₐ—(X)—(N)_{b}-3′ wherein 0>a>15, 0>b>15 N is any nucleotide, and X is a fragment of an adhesion-specific gene.

19. The nucleic acid of claim 18, wherein X comprises at least 8 nucleotides.

20. A Neisseria bacterium in which one or more adhesion-specific gene (s) has been knocked out.

21. The bacterium of claim 20, wherein knocked-out gene has a mutation in its coding region or in its transcriptional control regions.

22. The bacterium of claim 20, wherein the level of mRNA transcribed from the adhesion-specific gene (s) is <1% of that produced by a corresponding wild-type bacterium.

23. A mutant protein, comprising the amino acid sequence of an adhesion-specific protein, or a fragment thereof, but wherein one or more amino acids of said amino acid sequence is/are mutated.

24. The mutant protein of claim 23, wherein the amino acids which is/are mutated result in the reduction or removal of an activity of the adhesion-specific protein which is responsible directly or indirectly for adhesion to epithelial cells.

25. A nucleic acid encoding the protein of claim 23 or claim 24.

26. A method for producing the nucleic acid of claim 25, comprising the steps of: (a) providing source nucleic acid encoding an adhesion-specific gene, and (b) performing mutagenesis on the source nucleic acid to provide nucleic acid encoding the mutant protein of claim 23.

27. A pharmaceutical composition comprising an agent selected from the group consisting of the compound of claim 12, the antibody of claim 14 or claim 15, the nucleic acid of any one of claims 16 to 19, the bacterium of claim 20 or claim 22, the mutant protein of claim 23 or claim 24, and the nucleic acid of claim 25.

28. The method of claim 1, wherein the Neisserial cell is N. memingitidis.

29. The method of claim 1, wherein the epithelial cell is a human nasopharynx cell.

30. The method of claim 1, wherein the adhesion-specific protein is set out in Table I or Table II.

31. The method of claim 1, wherein the adhesion-specific protein is set out in Table III or Table V.
